(19) United States
(12) Patent Application Publication
(10) Pub. No.: US 2026/0327217 A1
(43) Pub. Date: Oct. 22, 2026

(54) THIAZOLE DERIVATIVES AS TAU AGGREGATION INHIBITORS

(71) Applicant: WisTa Laboratories Ltd., Singapore (SG)

(72) Inventors: Claude Michel Wischik, Aberdeen, Aberdeenshire (GB); Michael Philip Mazanetz, Newlands, Glasgow (GB); Louise Charlotte Serpell, Falmer Sussex (GB); Soumya Palliyil Soman, Aberdeen, Aberdeenshire (GB); Steven John Kemp, Old Aberdeen, Aberdeenshire (GB); Michael Simpson, Old Aberdeen, Aberdeenshire (GB); John Mervyn David Storey, Old Aberdeen, Aberdeenshire (GB)

(73) Assignee: WisTa Laboratories Ltd., Singapore (SG)

(21) Appl. No.: 19/113,758

(22) PCT Filed: Sep. 8, 2023
(86) PCT No.: PCT/EP2023/074788

§ 371 Date: Mar. 20, 2025

(30) Foreign Application Priority Data
Sep. 21, 2022 (GB) 2213796.2

(51) Int. Cl. C07D 417/12; A61K 31/426; A61K 31/427; C07D 277/56; C07D 417/14

(52) U.S. Cl. CPC C07D 417/12; A61K 31/426; A61K 31/427; C07D 277/56; C07D 417/14

(57) ABSTRACT

The present invention provides compounds of general formula (I): or pharmaceutically acceptable salts, solvates or hydrates thereof, wherein R^{N} is independently —H or -Me; R^{X} is independently C₁₋₄ alkyl, optionally substituted with halo or hydroxy; —NHR^{N1}, wherein R^{N1} is C₁₋₄ alkyl; or C₅₋₁₀ heteroaryl; R^{Y} is independently: C₅₋₁₀ heteroaryl, optionally substituted with methyl, halo or phenyl; or neopentyl. The compounds are of use as tau aggregation inhibitors (TAIs).

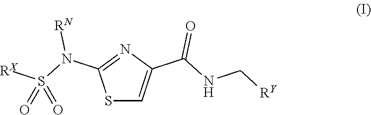

### FIELD OF THE INVENTION

The present invention relates primarily to compounds with activity as tau aggregation inhibitors (TAIs) and to their use in methods of treatment of tauopathies including, but not limited to, Alzheimer's disease.

### BACKGROUND

Tau protein aggregation forming neurofibrillary tangles and neuritic plaques are correlated with cognitive decline in Alzheimer's disease (AD) [1-3]. There is therefore interest in developing a treatment targeting this pathology. Hydromethylthionine mesylate (HMTM; previously referred to as leuco-methylthionine mesylate, LMTM) has been shown to have exposure-dependent pharmacological activity on clinical decline and brain atrophy in both AD and frontotemporal dementia [4, 5].

A core tau fragment corresponding to one of the species isolated from proteolytically stable AD paired helical filaments (PHFs) comprising residues 297-391 (dGAE) assembles spontaneously in the absence of polyanionic cofactors in vitro to form PHFs identical to native PHFs isolated from brain tissues [6-9]. Hydromethylthionine (HMT) inhibits assembly of dGAE filaments in vitro at a protein: HMT stoichiometric ratio of 1:0.1 [10].

The PHF core isolated from AD brain tissues is an extremely stable structure which resists proteolysis and requires harsh solvents, such as formic acid, to release the constituent tau protein [11] Assembly of tau aggregates is an autocatalytic process in which the core tau oligomer acts to nucleate further conversion of normal full-length tau into a truncated toxic species which progressively degrades neuronal function [12, 13]. The binding affinity for tau capture in vitro is on the order of 20 nM, a value that is substantially higher than that of physiological tau-tubulin binding (~400 nM; [14]). This drives the redistribution of the tau protein pool from normal soluble species to pathologically assembled forms [15].

Despite the availability of the structure of the AD PHF core and the core of the filaments present in Pick's disease at atomic resolution [16, 17], the elucidation of the molecular mechanisms of pathological assembly of tau and other proteins in neurodegenerative diseases remains elusive [18-20]. A number of precursors and oligomeric states have been identified for some proteins [21-24]. However, isolation and further characterisation of these structurally heterogeneous and transient forms remains a challenge, since it is unclear how they could be isolated and made amenable to unbiased structural and biochemical analysis.

The present inventors have previously reported that HMT has clinical pharmacological activity at an estimated steady state brain concentration of about 0.1 μM; which is of the same order as the EC₅₀ value of 0.6 μM seen in the cell assay [4, 13].

Given the intractability of the PHF core, it has not appeared possible to design small molecule TAIs which could be expected to act on an intracellular target in the brain at concentrations which are safe and tolerable clinically.

The present invention has been devised in light of the above considerations.

### SUMMARY OF THE INVENTION

The present inventors have used a combination of molecular dynamics (MD) simulation, immunochemistry, biochemistry and medicinal chemistry to investigate the molecular mechanisms of monomer capture by the PHF core oligomer and how HMT binds to the core tau unit of the PHF. They have shown that assembly initiation depends on a sequence of steps involving first the anchoring of the monomer and then the subsequent unravelling of the core unit to permit the stable cross-sheet structure of the PHF core to form. HMT works by binding to a cryptic druggable pocket within the core unit which stabilises it in a compact conformation that cannot participate in the unravelling required for further self-assembly.

The cryptic binding pocket identified by the present inventors provided a basis for defining a pharmacophore model and this model was used to design alternative tau aggregation inhibitors (TAIs). A set of compounds unrelated chemically to HMT were synthesised to test whether new inhibitors could be designed rationally to act at the druggable pocket and to optimise some of the medicinal chemistry features required for inhibitory activity. New inhibitors were found which have activity in a cell-based aggregation assay described previously [13].

Accordingly, in one aspect the present invention provides novel compounds, useful as tau aggregation inhibitors. The compounds contain a thiazole core and are of general formula:

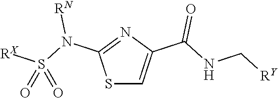

wherein R^{N} is independently —H or -Me;
R^{X} is independently C₁₋₄ alkyl, optionally substituted with halo or hydroxy; —NHR^{N1}, wherein R^{N1} is C₁₋₄ alkyl; or C₅₋₁₀ heteroaryl;
R^{Y} is independently: C₅₋₁₀ heteroaryl, optionally substituted with methyl, halo or phenyl; or neopentyl.

Also encompassed by the present invention are pharmaceutically acceptable salts, solvates and hydrates of these compounds.

The invention further relates to methods, uses, compositions and other materials employing these compounds as tau protein aggregation inhibitors and as therapeutics or prophylactics of diseases associated with tau protein aggregation (“tauopathies”). The invention further provides processes for making these compounds.

The invention includes the combination of the aspects and preferred features described except where such a combination is clearly impermissible or expressly avoided.

### SUMMARY OF THE FIGURES

Embodiments and experiments illustrating the principles of the invention will now be discussed with reference to the accompanying figures in which:

FIG. 1. Structures of compounds for which their activity in a cell-based tau aggregation assay has been tested. The core thiazole moiety is shown in the middle, the sulfonamide substituents, labelled 1-6, are shown above, and the amide substituents, numbered 7-12, are shown at the bottom of the schema.

FIG. 2. Inhibition of tau aggregation in fibroblasts transfected with inducible full-length tau1-441 and constitutive expression of truncated tau (residues 295-391) [13, 25] for (A) MT and (B) Compound 9. Using this data, the concentration at which there is 50% inhibition of the lower 12-kDa band (EC₅₀ value) was 0.6 μM and 3.65 μM for MT and Compound 9, respectively (results for the assay in replicate are tabulated below in Example 3; the mean value for Compound 9 was 4.816 μM).

FIG. 3. The layering assembly. of the critical fragment (residues 306-378) of monomeric dGAE sequences (cartoon) onto a single layer of a PHF stack (main-chain lines). (A) The approach of the hairpin loop residues 337-355 (recognised by 1D12) during the start of the assembly, Frame 1. (B) Frame 44 shows the unravelling of stable monomer and the extension of the C- and N-termini. (C) Frame 75 shows further interactions of the monomer just prior to flipping of Pro332. (D) Frame 143 shows the truncated dGAE monomer incorporated onto the PHF stack. The sequence of the truncated dGAE monomer used in the Nudged Elastic band (NEB) simulation is shown at the bottom of the figure along with the regions which map to the epitopes recognised by a panel of single chain antibodies (scAbs; in order of decreasing shades of grey: CE2>1D12>1G2>CA4).

FIG. 4. The HMT-tau binding pose of an HMT-protein complex. The sequence of the truncated tau residues 295-391 used in the MD simulation is shown at the bottom of the figure along with the regions which map to the epitopes recognised by the corresponding scab (in order of decreasing shades of grey: CE2>E2E8>1D12>1G2>CA4.

FIG. 5. Immunoreactivity of core region scAbs to dGAE assembled in the presence or absence of TAIs HMT and compound 9. dGAE samples were presented for scAb binding in the solution phase by first capturing dGAE with mAb 423 and then detecting bound dGAE with scAbs. The mAb 423 specifically recognises the Glu391-dependent C-terminus of the dGAE fragment. The binding profiles for scAbs (A) CA4, (B) 1G2, (C) CE2, and (D) 1D12 were obtained using an anti-human C kappa HRP-conjugated secondary antibody. Reactivity with pre-assembly soluble dGAE (dGAE 0 h) was included to show the maximal reactivity of scAbs to non-aggregated dGAE. Reactivity of dGAE was measured after 24 h assembly without addition (dGAE 24 h) or in the presence of HMT or compound 9 (C9); compound 9 was dissolved in DMSO for which there is also a solvent control. Values are expressed as absorbance at 450 nm (mean±SE; n=2; samples run in duplicate).

FIG. 6. Immunoblot intensity analysis shows progressive loss of binding of recombinant scAbs to dGAE (tau297-391) epitopes over the course of 8 hours assembly in vitro. Signal intensity of dots at each time point was measured using FIJI and background subtracted. To normalise, where there was signal observed at time 0 in a blot, this was defined as 1, with remaining time points quantified relative to this. In some experimental replicates, no signal was observed. These results were incorporated into the final pooled data shown, resulting in time 0 values of <1.0 for antibodies CE2, CA4 and E2E8, where 3/9, 4/8 and 7/8 replicates, respectively, gave positive results. Where there was no signal on a blot this was designated as zero and the intensity for each time point is expressed as mean±SEM. Exposure times were used that ensured signals were not saturating and within linear range.

FIG. 7. Schematic overview of computer-aided drug design (CADD) methods.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

Hydromethylthionine (HMT) is the active moiety in hydromethylthionine mesylate (HMTM), and is a potent tau aggregation inhibitor. Hydromethylthionine (HMT) may also be referred to as leucomethylthioninium (LMT). For the avoidance of doubt, these two terms are synonymous and may be used interchangeably herein.

The present inventors have used a combination of molecular dynamics (MD) simulation, immunochemistry, biochemistry and medicinal chemistry to investigate the molecular mechanisms of monomer capture by the PHF core oligomer and how HMT binds to the core tau unit of the PHF.

Molecular dynamics offers the advantage of providing a means of exploring extensive conformational spaces and determining the inherent heterogeneity and stability of specific states of the molecule. Although obtaining direct structural proof for the existence of certain conformations is difficult, particularly for a molecule with the inherent ability to aggregate spontaneously as is the case for dGAE, it is possible to make structural predictions which can be tested using immunochemistry, medicinal chemistry and biochemistry. Several tools are essential to make this approach feasible: model systems in which the assembly of the PHF core can be monitored, a family of specific monoclonal antibodies which recognise epitopes located in the PHF core domain and a prototype inhibitor which can be used as a basis for defining a pharmacophore model for targeted medicinal chemistry. These techniques were applied to investigate both the mode of assembly of the PHF core and the pharmacological inhibition of assembly.

An initial MD analysis of the mode of monomer capture by an assembled PHF core template can be summarised in a series of sequential stages. First, the monomer is anchored to the template through the tight hairpin loop formed by residues 337-355, then Pro332 undergoes a switch in conformation which permits unfolding of the monomer and zipping of its N- and C-terminal arms with corresponding segments of the existing assembly. These latter events occur over a short time period with residues 319-331 binding in a direction from C- to N-terminus, and residues 355-367 binding in the N- to C-terminal direction. This folding sequence is supported by progressive loss of immunoreactivity of a panel of scAbs recognising epitopes spanning the PHF core tau unit. Immunoreactivity with CE2 and CA4, which recognise epitopes immediately adjacent to the hairpin loop, was lost very early, whereas occlusion of epitopes recognised by 1G2 and E2E8 located nearer to the C-terminus occurred at a later stage. Although it has been proposed that a short fragment comprising residues 306-311 triggers tau assembly [26], the present analysis shows that the process of templated monomer capture occurs over a more extended distance than can be modelled by short peptides in vitro. We show that initial capture occurs through the highly charged hairpin loop corresponding to residues 337-355. This is followed by zipping up of the N- and C-terminal segments onto the pre-existing template. In this sequence, the N- and C-terminal arms of the core tau unit need to be flexible to permit formation of the stable crossed β-sheet structure that is characteristic of the PHF core.

Since HMT is known to be a potent inhibitor of dGAE assembly, the present inventors used it as a molecular probe to determine whether transiently stable cryptic ligand-binding pockets exist that could be used for structure-based drug design. Of the 750,000 possible protein conformations of dGAE identified from the MD simulations, they were able to identify a single complex where HMT remained tightly bound to the protein structure and did not vary greatly over 100 ns of MD simulation time. Comparison with simulations in the absence of HMT revealed that HMT acts by stabilising one of the conformations that is available among the ensemble of possible conformations of the core tau unit. The driving force responsible for the wrapped-up assembly-incompetent conformation is the formation of intra-molecular hydrogen bonds and hydrophobic collapse which reduces the water accessible, polar and hydrophobic surface areas required for alignment and accommodation of the monomer onto a pre-existing oligomer. We do not know whether a different class of TAIs might be able to act by stabilising a different conformation of the core tau unit. We can however conclude from the present study that HMT acts by stabilising a conformation that competes with the unravelling of the N- and C-terminal domains of the monomer that is required for oligomer elongation.

The cryptic binding pocket identified provides a basis for defining a pharmacophore model. A detailed discussion of the molecular dynamics studies and definition of a pharmacophore model is set out in WO2022/008545 (WisTa Laboratories Ltd.), the disclosure of which is hereby incorporated by reference in its entirety.

The present study provides an explanation for the surprising clinical potency of HMT as a TAI. In essence, HMT acts by stabilising an endogenous assembly-incompetent conformation of the PHF core tau unit. The inventors have shown that the druggable pocket bound by HMT within the PHF core provides a basis for rational design of chemically unrelated TAIs.

The present inventors have now designed alternative TAIs, based on the aforementioned pharmacophore model. These have been synthesised and tested using two biological assay systems. The HMT-binding pocket is predominantly hydrophobic in nature with some sites suitable for forming hydrogen bonds. A set of compounds unrelated chemically to HMT was synthesised, to test whether new inhibitors could be designed rationally to act at the druggable pocket and to optimise some of the medicinal chemistry features required for inhibitory activity.

Several new inhibitors were found which have activity in a cell-based aggregation assay reported previously [13]. The best of these was compared with HMT in a new aqueous-phase immunoassay examining aggregation-dependent epitope occlusion with respect to a panel of antibodies.

Accordingly, in one aspect the present invention provides a compound of general formula:

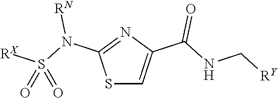

wherein R^{N} is independently —H or -Me;
R^{X} is independently C₁₋₄ alkyl, optionally substituted with halo or hydroxy; —NHR^{N1}, wherein R^{N1} is C₁₋₄ alkyl; or C₅₋₁₀ heteroaryl;
R^{Y} is independently: C₅₋₁₀ heteroaryl, optionally substituted with methyl, halo or phenyl; or neopentyl;
or a pharmaceutically acceptable salt, solvate, or hydrate thereof.

In some embodiments, R^{N} is —H.

In some embodiments, R^{N} is -Me.

In some embodiments, R^{X} is independently C₁₋₄ alkyl, optionally substituted with halo or hydroxy.

In some embodiments, R^{X} is -Me, -Et, -nPr, -iPr, -nBu, -iBu, or -tBu, optionally substituted with halo or hydroxy.

In some embodiments, R^{X} is -Me, -Et, -nPr, or -iPr, optionally substituted with halo or hydroxy.

In some embodiments, R^{X} is -Me or -Et, optionally substituted with halo or hydroxy.

In some embodiments, R^{X} is -Me, optionally substituted with halo.

In some embodiments, R^{X} is -Me, optionally substituted with —F or —Cl.

In some embodiments, R^{X} is —CH₂F.

In some embodiments, R^{X} is unsubstituted -Me.

In some embodiments, R^{X} is -Et, optionally substituted with hydroxy.

In some embodiments, R^{X} is —CH₂CH₂OH.

In some embodiments, R^{X} is independently —NHR^{N1}, wherein R^{N1} is C₁₋₄ alkyl.

In some embodiments, R^{N1} is -Me, -Et, -nPr, -iPr, -nBu, -iBu, or -tBu.

In some embodiments, R^{N1} is -Me, -Et, -nPr, or -iPr.

In some embodiments, R^{N1} is -Me, or -Et.

In some embodiments, R^{X} is independently —NHMe

In some embodiments, R^{X} is independently C₅₋₁₀ heteroaryl.

In some embodiments, R^{X} is a nitrogen-containing C₅₋₁₀ heteroaryl group.

In some embodiments, R^{X} is selected from indolyl, benzimidazolyl, pyrollyl, imidazolyl, pyrazolyl.

In some embodiments, R^{X} is an indolyl group.

In some embodiments, R^{X} is

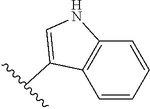 [SMILES: *c1c[nH]c2ccccc12]

In some embodiments, R^{Y} is neopentyl (—CH₂C(CH₃)₃).

In some embodiments R^{Y} is independently: C₅₋₁₀ heteroaryl, optionally substituted with methyl, halo or phenyl.

In some embodiments, R^{Y} is a nitrogen-containing C₅₋₁₀ heteroaryl, optionally substituted with methyl, halo or phenyl.

In some embodiments, R^{Y} is a nitrogen-containing C₅₋₁₀ heteroaryl, optionally substituted with halo or phenyl.

In some embodiments, R^{Y} is a nitrogen-containing C₅₋₁₀ heteroaryl, optionally substituted with -Me, —Cl or -Ph.

In some embodiments, R^{Y} is a nitrogen-containing C₅₋₁₀ heteroaryl selected from pyrrolyl, imidazolyl, pyrazolyl, indolyl, and benzimidazolyl, optionally substituted with -Me, —Cl or -Ph.

In some embodiments, R^{Y} is a nitrogen-containing C₅₋₁₀ heteroaryl selected from pyrrolyl, pyrazolyl, and benzimidazolyl, optionally substituted with -Me, —Cl or -Ph.

In some embodiments, R^{Y} is unsubstituted C₅₋₁₀ heteroaryl.

In some embodiments, R^{Y} is an unsubstituted nitrogen-containing C₅₋₁₀ heteroaryl.

In some embodiments, R^{Y} is unsubstituted pyrrolyl, imidazolyl, pyrazolyl, indolyl, or benzimidazolyl.

In some embodiments, R^{Y} is unsubstituted pyrrolyl or indolyl.

In some embodiments, R^{Y} is selected from:

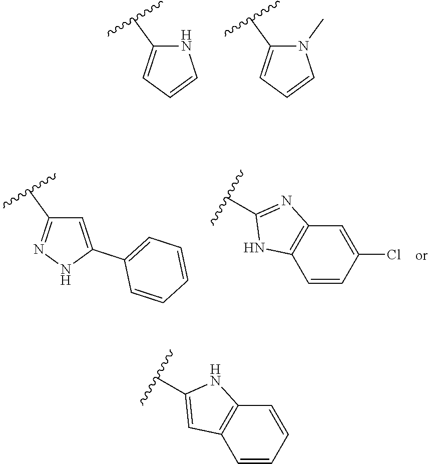

### Preferred Compounds

In general, the present invention relates to one or more compounds selected from the following compounds, and their use in medicine:

| Compound | Structure |
|---|---|
| 1 | 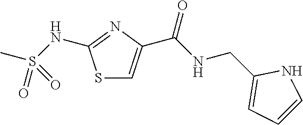 [SMILES: CS(=O)(=O)Nc1nc(C(=O)NCc2ccc[nH]2)cs1] |
| 2 | 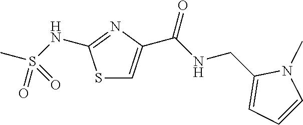 |
| 3 | 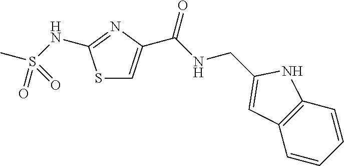 [SMILES: CS(=O)(=O)Nc1nc(C(=O)NCc2cc3ccccc3[nH]2)cs1] |
| 4 | 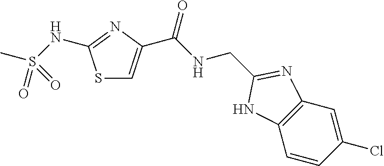 [SMILES: CS(=O)(=O)Nc1nc(C(=O)NCc2nc3cc(Cl)ccc3[nH]2)cs1] |
| 5 | 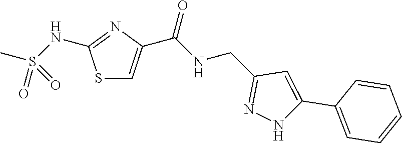 [SMILES: CS(=O)(=O)Nc1nc(C(=O)NCc2cc(-c3ccccc3)[nH]n2)cs1] |
| 6 | 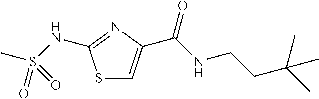 [SMILES: CC(C)(C)CCNC(=O)c1csc(NS(C)(=O)=O)n1] |
| 7 | 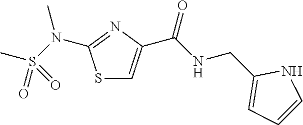 [SMILES: CN(c1nc(C(=O)NCc2ccc[nH]2)cs1)S(C)(=O)=O] |
| 8 | 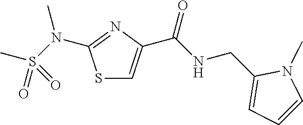 |
| 9 | 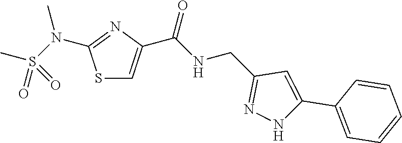 [SMILES: CN(c1nc(C(=O)NCc2cc(-c3ccccc3)[nH]n2)cs1)S(C)(=O)=O] |
| 10 | 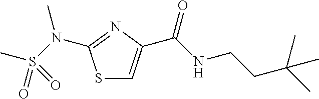 [SMILES: CN(c1nc(C(=O)NCCC(C)(C)C)cs1)S(C)(=O)=O] |
| 11 | 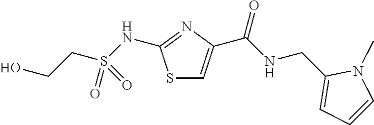 [SMILES: Cn1cccc1CNC(=O)c1csc(NS(=O)(=O)CCO)n1] |
| 12 | 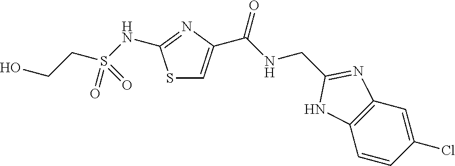 [SMILES: O=C(NCc1nc2cc(Cl)ccc2[nH]1)c1csc(NS(=O)(=O)CCO)n1] |
| 13 | 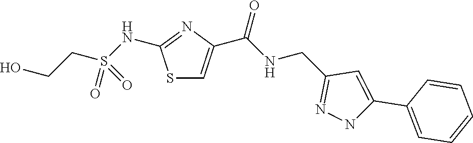 [SMILES: O=C(NCc1cc(-c2ccccc2)[nH]n1)c1csc(NS(=O)(=O)CCO)n1] |
| 14 | 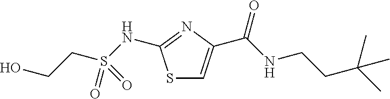 [SMILES: CC(C)(C)CCNC(=O)c1csc(NS(=O)(=O)CCO)n1] |
| 15 | 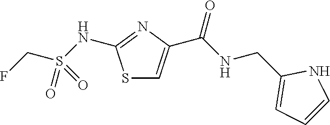 [SMILES: O=C(NCc1ccc[nH]1)c1csc(NS(=O)(=O)CF)n1] |
| 16 | 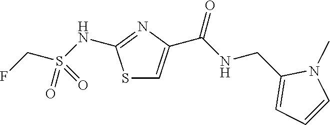 [SMILES: Cn1cccc1CNC(=O)c1csc(NS(=O)(=O)CF)n1] |
| 17 | 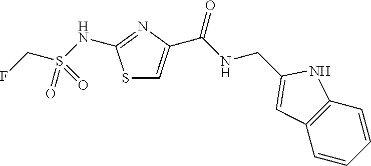 [SMILES: O=C(NCc1cc2ccccc2[nH]1)c1csc(NS(=O)(=O)CF)n1] |
| 18 | 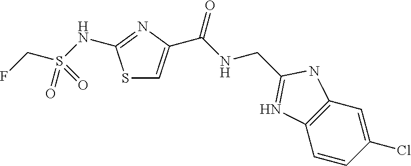 [SMILES: O=C(NCc1nc2ccc(Cl)cc2[nH]1)c1csc(NS(=O)(=O)CF)n1] |
| 19 | 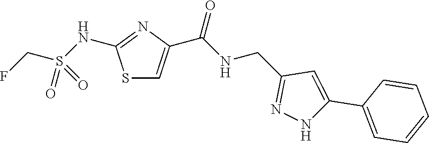 [SMILES: O=C(NCc1cc(-c2ccccc2)[nH]n1)c1csc(NS(=O)(=O)CF)n1] |
| 20 | 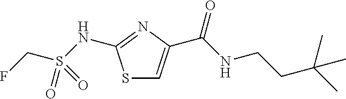 [SMILES: CC(C)(C)CCNC(=O)c1csc(NS(=O)(=O)CF)n1] |
| 21 | 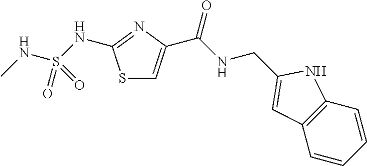 [SMILES: CNS(=O)(=O)Nc1nc(C(=O)NCc2cc3ccccc3[nH]2)cs1] |
| 22 | 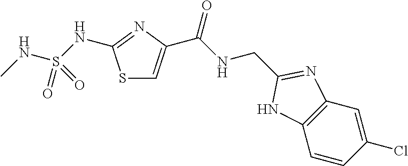 [SMILES: CNS(=O)(=O)Nc1nc(C(=O)NCc2nc3cc(Cl)ccc3[nH]2)cs1] |
| 23 | 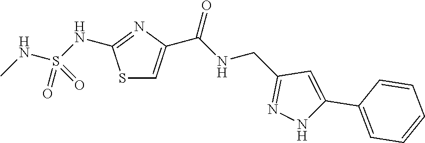 [SMILES: CNS(=O)(=O)Nc1nc(C(=O)NCc2cc(-c3ccccc3)[nH]n2)cs1] |
| 24 | 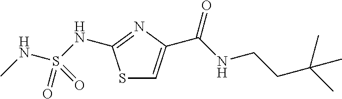 [SMILES: CNS(=O)(=O)Nc1nc(C(=O)NCCC(C)(C)C)cs1] |
| 25 | 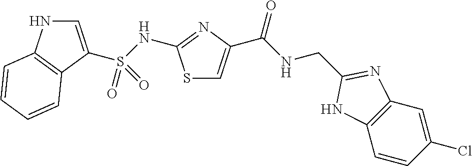 [SMILES: O=C(NCc1nc2cc(Cl)ccc2[nH]1)c1csc(NS(=O)(=O)c2c[nH]c3ccccc23)n1] |
| 26 | 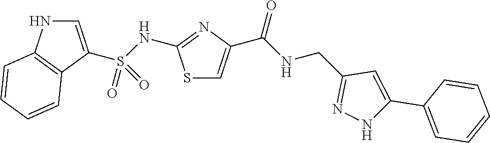 [SMILES: O=C(NCc1cc(-c2ccccc2)[nH]n1)c1csc(NS(=O)(=O)c2c[nH]c3ccccc23)n1] |

In this and all other aspects of the invention, unless context demands otherwise, a compound may be selected from the list consisting of 1, 2, 3, 4, 5, 6, 7, 8, 9, 10, 11, 12, 13, 14, 15, 16, 17, 18, 19, 20, 21, 22, 23, 24, 25, and 26.

In one embodiment, a compound may be selected from the list consisting of 1, 4, 5, 6, 7, 8, 9, 10, 11, 12, 15, 16, 18, 20, 21, and 23.

In one embodiment, the compound is selected from the list consisting of 1, 4, 5, 6, 9, 10, 12, 15, 16, and 20.

In one embodiment, the compound is selected from the list consisting of 5, 9, 10, 12, and 20.

In one embodiment, the compound is selected from the list consisting of 9, 10, 14, and 25.

In one embodiment, the compound is compound 9.

Preferred compounds of the present invention are those which show activity in the assays described herein. Particularly preferred compounds have an activity of less than 0.5 in the cell based aggregation inhibition assay described herein (ratio of truncated tau—full length tau; tested at 2 μM).

Preferably, the compounds have an EC₅₀ of less than 500, 250, 200, 100, or 50 as determined with reference to the Examples herein. Preferably, the compounds have a B₅₀ of less than 750, 500, 200, or 100 as determined with reference to the Examples herein.

### Isotopic Variation

In one embodiment, one or more of the carbon atoms of the compound is ¹¹C or ¹³C or ¹⁴C.

In one embodiment, one or more of the carbon atoms of the compound is ¹¹C.

In one embodiment, one or more of the carbon atoms of the compound is ¹³C.

In one embodiment, one or more of the carbon atoms of the compound is ¹⁴C.

In one embodiment, one or more of the nitrogen atoms of the compound is ¹⁵N.

### Uses to Reverse or Inhibit the Aggregation of Tau Protein.

One aspect of the invention is the use of a thiazole-containing compound to reverse or inhibit the aggregation of tau protein. This aggregation may be in vitro, or in vivo, and may be associated with a tauopathy disease state as discussed herein. Also provided are methods of reversing or inhibiting the aggregation of tau protein comprising contacting the aggregate or protein with a compound as described herein.

As discussed below, various tauopathy disorders that have been recognised which feature prominent tau pathology in neurons and/or glia and this term has been used in the art for several years. The similarities between these pathological inclusions and the characteristic tau inclusions in diseases such as AD indicate that the structural features are shared and that it is the topographic distribution of the pathology that is responsible for the different clinical phenotypes observed. In addition to specific diseases discussed below, those skilled in the art can identify tauopathies by combinations of cognitive or behavioural symptoms, plus additionally through the use of appropriate ligands for aggregated tau as visualised using PET or MRI, such as those described in WO2010/034982.

Methods of Treatment or Prophylaxis and 1^{st} & 2^{nd} Medical Uses

One aspect of the present invention pertains to a method of treatment or prophylaxis of a tauopathy condition in a patient, comprising administering to said patient a therapeutically-effective amount of a thiazole-containing compound, as described herein.

Aspects of the present invention relate to “tauopathies”. As well as Alzheimer's disease (AD), the pathogenesis of neurodegenerative disorders such as Pick's disease and Progressive Supranuclear Palsy (PSP) appears to correlate with an accumulation of pathological truncated tau aggregates in the dentate gyrus and stellate pyramidal cells of the neocortex, respectively. Other dementias include fronto-temporal dementia (FTD); FTD with parkinsonism linked to chromosome 17 (FTDP-17); disinhibition-dementia-parkinsonism-amyotrophy complex (DDPAC); pallido-ponto-nigral degeneration (PPND); Guam-amyotrophic lateral sclorosis syndrome; pallido-nigro-luysian degeneration (PNLD); cortico-basal degeneration (CBD); dementia with argyrophilic grains (AgD); dementia pugilistica (DP) wherein despite different topography, neurofibrillary tangles (NFTs) are similar to those observed in AD [27]; chronic traumatic encephalopathy (CTE), a tauopathy including DP as well as repeated and sports-related concussion [28]. Others are discussed in Wischik et al. 2000—see especially Table 5.1).

Abnormal tau in NFTs is found also in Down Syndrome (DS) and in Dementia with Lewy bodies (DLB) [32]. Tau-positive NFTs are also found in postencephalitic parkinsonism (PEP) [33]. Glial tau tangles are observed in subacute sclerosing panencephalitis (SSPE) [34].

Other tauopathies include Niemann-Pick disease type C(NPC) [35]; Sanfilippo syndrome type B (or mucopolysaccharidosis III B, MPS III B) [36]; myotonic dystrophies (DM), DM1 and DM2 [38].

Additionally, there is a growing consensus in the literature that a tau pathology may also contribute more generally to cognitive deficits and decline, including in mild cognitive impairment (MCI) (see e.g., [39]).

All of these diseases, which are characterised primarily or partially by abnormal tau aggregation, are referred to herein as “tauopathies” or “diseases of tau protein aggregation”.

In this and all other aspects of the invention relating to tauopathies, preferably the tauopathy is selected from the list consisting of the indications above, i.e., AD, Pick's disease, PSP, FTD, FTDP-17, DDPAC, PPND, Guam-ALS syndrome, PNLD, CBD, AgD, DS, SSPE, DP, PEP, SSPE, DLB, CTE and MCI.

In one preferred embodiment the tauopathy is Alzheimer's disease (AD).

One aspect of the present invention pertains to a compound as described herein, for use in a method of treatment or prophylaxis (e.g., of a tauopathy condition) of the human or animal body by therapy.

One aspect of the present invention pertains to use of compound as described herein, in the manufacture of a medicament for use in the treatment or prophylaxis of a tauopathy condition.

A further embodiment is a method of treatment or prophylaxis of a disease of tau protein aggregation as described herein, which method comprises administering to a subject a compound as described herein, or therapeutic composition comprising the same, such as to inhibit the aggregation of the tau protein associated with said disease state.

### Other Methods and Uses

In a further embodiment there is disclosed a compound as described herein, or therapeutic composition comprising the same, for use in a method of treatment or prophylaxis of a disease of tau protein aggregation as described above, which method comprises administering to a subject the thiazole-containing compound or composition such as to inhibit the aggregation of the tau protein associated with said disease state.

In a further embodiment there is disclosed use of a compound as described herein in the preparation of a medicament for use in a method of treatment or prophylaxis of a disease of tau protein aggregation as described above, which method comprises administering to a subject the medicament such as to inhibit the aggregation of the tau protein associated with said disease state.

In one embodiment there is disclosed a method of regulating the aggregation of a tau protein in the brain of a mammal, which aggregation is associated with a disease state as described above, the treatment comprising the step of administering to said mammal in need of said treatment, a prophylactically or therapeutically effective amount of an inhibitor of said aggregation, wherein the inhibitor is a thiazole-containing compound as described herein.

One aspect of the invention is a method of inhibiting production of protein aggregates (e.g., in the form of paired helical filaments (PHFs), optionally in neurofibrillary tangles (NFTs)) in the brain of a mammal, the treatment being as described herein.

In one aspect the invention provides a drug product for the treatment of a disease state associated with tau protein aggregation in a mammal suffering therefrom, comprising a container labelled or accompanied by a label indicating that the drug product is for the treatment of said disease, the container containing one or more dosage units each comprising at least one pharmaceutically acceptable excipient and, as an active ingredient, an isolated pure compound of the invention.

### Compositions, Formulations and Purity

In one embodiment, the compound may be provided or used in a composition which is equal to or less than 100, 99, 98, 97, 96, 95, 94, 93, 92, 91, or 90% pure.

One aspect of the present invention pertains to a dosage unit (e.g., a pharmaceutical tablet or capsule) comprising 20 to 300 mg of a compound as described herein (e.g., obtained by, or obtainable by, a method as described herein; having a purity as described herein; etc.), and a pharmaceutically acceptable carrier, diluent, or excipient.

In one embodiment, the dosage unit is a tablet.

In one embodiment, the dosage unit is a capsule.

Dosage units (e.g., a pharmaceutical tablet or capsule) comprising 20 to 300 mg of a compound as described herein and a pharmaceutically acceptable carrier, diluent, or excipient are discussed in more detail hereinafter.

In one embodiment, the amount is 30 to 200 mg.

In one embodiment, the amount is about 25 mg.

In one embodiment, the amount is about 35 mg.

In one embodiment, the amount is about 50 mg.

In one embodiment, the amount is about 70 mg.

In one embodiment, the amount is about 125 mg.

In one embodiment, the amount is about 175 mg.

In one embodiment, the amount is about 250 mg.

In one embodiment, the pharmaceutically acceptable carrier, diluent, or excipient is or comprises one or both of a glyceride (e.g., Gelucire 44/14®; lauroyl macrogol-32 glycerides PhEur, USP) and colloidal silicon dioxide (e.g., 2% Aerosil 200®; Colloidal Silicon Dioxide PhEur, USP).

### Formulations

While it is possible for the compound to be used (e.g., administered) alone, it is often preferable to present it as a composition or formulation.

In one embodiment, the composition is a pharmaceutical composition (e.g., formulation, preparation, medicament) comprising a thiazole-containing compound, as described herein, and a pharmaceutically acceptable carrier, diluent, or excipient.

In one embodiment, the composition is a pharmaceutical composition comprising at least one compound as described herein, together with one or more other pharmaceutically acceptable ingredients well known to those skilled in the art, including, but not limited to, pharmaceutically acceptable carriers, diluents, excipients, adjuvants, fillers, buffers, preservatives, anti-oxidants, lubricants, stabilisers, solubilisers, surfactants (e.g., wetting agents), masking agents, colouring agents, flavouring agents, and sweetening agents.

In one embodiment, the composition further comprises other active agents, for example, other therapeutic or prophylactic agents.

Suitable carriers, diluents, excipients, etc. can be found in standard pharmaceutical texts. See, for example, Handbook of Pharmaceutical Additives, 2nd Edition (eds. M. Ash and I. Ash), 2001 (Synapse Information Resources, Inc., Endicott, New York, USA), Remington's Pharmaceutical Sciences, 20th edition, pub. Lippincott, Williams & Wilkins, 2000; and Handbook of Pharmaceutical Excipients, 2nd edition, 1994.

Another aspect of the present invention pertains to methods of making a pharmaceutical composition comprising admixing at least one [¹¹C]-radiolabelled thiazole-containing compound, as defined herein, together with one or more other pharmaceutically acceptable ingredients well known to those skilled in the art, e.g., carriers, diluents, excipients, etc. If formulated as discrete units (e.g., tablets, etc.), each unit contains a predetermined amount (dosage) of the active compound.

The term “pharmaceutically acceptable,” as used herein, pertains to compounds, ingredients, materials, compositions, dosage forms, etc., which are, within the scope of sound medical judgment, suitable for use in contact with the tissues of the subject in question (e.g., human) without excessive toxicity, irritation, allergic response, or other problem or complication, commensurate with a reasonable benefit/risk ratio. Each carrier, diluent, excipient, etc. must also be “acceptable” in the sense of being compatible with the other ingredients of the formulation.

The formulations may be prepared by any methods well known in the art of pharmacy. Such methods include the step of bringing into association the active compound with a carrier which constitutes one or more accessory ingredients. In general, the formulations are prepared by uniformly and intimately bringing into association the active compound with carriers (e.g., liquid carriers, finely divided solid carrier, etc.), and then shaping the product, if necessary.

The formulation may be prepared to provide for rapid or slow release; immediate, delayed, timed, or sustained release; or a combination thereof.

Formulations suitable for parenteral administration (e.g., by injection), include aqueous or non-aqueous, isotonic, pyrogen-free, sterile liquids (e.g., solutions, suspensions), in which the active ingredient is dissolved, suspended, or otherwise provided (e.g., in a liposome or other microparticulate). Such liquids may additionally contain other pharmaceutically acceptable ingredients, such as anti-oxidants, buffers, preservatives, stabilisers, bacteriostats, suspending agents, thickening agents, and solutes which render the formulation isotonic with the blood (or other relevant bodily fluid) of the intended recipient. Examples of excipients include, for example, water, alcohols, polyols, glycerol, vegetable oils, and the like. Examples of suitable isotonic carriers for use in such formulations include Sodium Chloride Injection, Ringer's Solution, or Lactated Ringer's Injection. Typically, the concentration of the active ingredient in the liquid is from about 1 ng/ml to about 10 μg/ml, for example from about 10 ng/ml to about 1 μg/ml. The formulations may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules, and tablets.

### Dosage

It will be appreciated by one of skill in the art that appropriate dosages of the compound, and compositions comprising the compound, can vary from patient to patient. Determining the optimal dosage will generally involve the balancing of the level of therapeutic benefit against any risk or deleterious side effects. The selected dosage level will depend on a variety of factors including, but not limited to, the activity of the particular compound, the route of administration, the time of administration, the rate of excretion of the compound, the duration of the treatment, other drugs, compounds, and/or materials used in combination, the severity of the condition, and the species, sex, age, weight, condition, general health, and prior medical history of the patient. The amount of compound and route of administration will ultimately be at the discretion of the physician, veterinarian, or clinician, although generally the dosage will be selected to achieve local concentrations at the site of action which achieve the desired effect without causing substantial harmful or deleterious side-effects.

Administration can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell(s) being treated, and the subject being treated. Single or multiple administrations can be carried out with the dose level and pattern being selected by the treating physician, veterinarian, or clinician.

In general, a suitable dose of the active compound is in the range of about 100 ng to about 25 mg (more typically about 1 μg to about 10 mg) per kilogram body weight of the subject per day. Where the active compound is a salt, an ester, an amide, a prodrug, or the like, the amount administered is calculated on the basis of the parent compound and so the actual weight to be used is increased proportionately.

In one embodiment, the active compound is administered to a human patient according to the following dosage regime: about 100 mg, 3 times daily.

In one embodiment, the active compound is administered to a human patient according to the following dosage regime: about 150 mg, 2 times daily.

In one embodiment, the active compound is administered to a human patient according to the following dosage regime: about 200 mg, 2 times daily.

However, in one embodiment, the compound is administered to a human patient according to the following dosage regime: about 50 or about 75 mg, 3 or 4 times daily.

In one embodiment, the compound is administered to a human patient according to the following dosage regime: about 100 or about 125 mg, 2 times daily.

### Preferred Combination Therapies

Combination treatments and therapies, in which two or more treatments or therapies are combined, for example, sequentially or simultaneously, are discussed in more detail hereinafter. Thus it will be understood that any of the medical uses or methods described herein may be used in a combination therapy.

In one embodiment, a treatment of the invention (e.g., employing a compound of the invention) is in combination with a cholinesterase inhibitor such as donepezil (Aricept™), rivastigmine (Exelon™) or galantamine (Reminyl™).

In one embodiment, a treatment of the invention (e.g., employing a compound of the invention) is in combination with an NMDA receptor antagonist such as memantine (Ebixa™, Namenda™).

In one embodiment, a treatment of the invention (e.g., employing a compound of the invention) is in combination with a muscarinic receptor agonist.

In one embodiment, a treatment of the invention (e.g., employing a compound of the invention) is in combination with an inhibitor of amyloid precursor protein to beta-amyloid (e.g., an inhibitor of amyloid precursor protein processing that leads to enhanced generation of beta-amyloid).

### Ligands and Labels

Thiazole-containing compounds discussed herein that are capable of inhibiting the aggregation of tau protein will also be capable of acting as ligands or labels of tau protein (or aggregated tau protein). Thus, in one embodiment, the thiazole-containing compound is a ligand of tau protein (or aggregated tau protein).

Such thiazole-containing compounds (ligands) may incorporate, be conjugated to, be chelated with, or otherwise be associated with, other chemical groups, such as stable and unstable detectable isotopes, radioisotopes, positron-emitting atoms, magnetic resonance labels, dyes, fluorescent markers, antigenic groups, therapeutic moieties, or any other moiety that may aid in a prognostic, diagnostic or therapeutic application.

For example, as noted above, in one embodiment, the compound is as defined above, but with the additional limitation that the compound incorporates, is conjugated to, is chelated with, or is otherwise associated with one or more (e.g., 1, 2, 3, 4, etc.) isotopes, radioisotopes, positron-emitting atoms, magnetic resonance labels, dyes, fluorescent markers, antigenic groups, or therapeutic moieties.

In one embodiment, the compound is a ligand as well as a label, e.g., a label for tau protein (or aggregated tau protein), and incorporates, is conjugated to, is chelated with, or is otherwise associated with, one or more (e.g., 1, 2, 3, 4, etc.) detectable labels.

For example, in one embodiment, the compound is as defined above, but with the additional limitation that the compound incorporates, is conjugated to, is chelated with, or is otherwise associated with, one or more (e.g., 1, 2, 3, 4, etc.) detectable labels.

Labelled compounds (e.g., when ligated to tau protein or aggregated tau protein) may be visualised or detected by any suitable means, and the skilled person will appreciate that any suitable detection means as is known in the art may be used.

For example, the thiazole-containing compound (ligand-label) may be suitably detected by incorporating a positron-emitting atom (e.g., ¹¹C) (e.g., as a carbon atom of one or more alkyl group substituents, e.g., methyl group substituents) and detecting the compound using positron emission tomography (PET) as is known in the art.

### Treatment

The term “treatment,” as used herein in the context of treating a condition, pertains generally to treatment and therapy, whether of a human or an animal (e.g., in veterinary applications), in which some desired therapeutic effect is achieved, for example, the inhibition of the progress of the condition, and includes a reduction in the rate of progress, a halt in the rate of progress, regression of the condition, amelioration of the condition, and cure of the condition. Treatment as a prophylactic measure (i.e., prophylaxis, prevention) is also included.

The term “therapeutically-effective amount,” as used herein, pertains to that amount of an active compound, or a material, composition or dosage form comprising an active compound, which is effective for producing some desired therapeutic effect, commensurate with a reasonable benefit/risk ratio, when administered in accordance with a desired treatment regimen.

Similarly, the term “prophylactically-effective amount,” as used herein, pertains to that amount of an active compound, or a material, composition or dosage form comprising an active compound, which is effective for producing some desired prophylactic effect, commensurate with a reasonable benefit/risk ratio, when administered in accordance with a desired treatment regimen.

The term “treatment” includes combination treatments and therapies, in which two or more treatments or therapies are combined, for example, sequentially or simultaneously. Examples of treatments and therapies include, but are not limited to, chemotherapy (the administration of active agents, including, e.g., drugs, antibodies (e.g., as in immunotherapy), prodrugs (e.g., as in photodynamic therapy, gene-directed enzyme prodrug therapy (GDEPT), antibody-directed enzyme prodrug therapy (ADEPT), etc.); surgery; radiation therapy; and gene therapy.

### Routes of Administration

The thiazole-containing compound, or a pharmaceutical composition comprising it, may be administered to a subject/patient by any convenient route of administration, whether systemically/peripherally or topically (i.e., at the site of desired action).

Routes of administration include, but are not limited to, oral (e.g., by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eyedrops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal (including, e.g., intracatheter injection into the brain); by implant of a depot or reservoir, for example, subcutaneously or intramuscularly.

### The Subject/Patient

The subject/patient may be an animal, mammal, a placental mammal, a marsupial (e.g., kangaroo, wombat), a monotreme (e.g., duckbilled platypus), a rodent (e.g., a guinea pig, a hamster, a rat, a mouse), murine (e.g., a mouse), a lagomorph (e.g., a rabbit), avian (e.g., a bird), canine (e.g., a dog), feline (e.g., a cat), equine (e.g., a horse), porcine (e.g., a pig), ovine (e.g., a sheep), bovine (e.g., a cow), a primate, simian (e.g., a monkey or ape), a monkey (e.g., marmoset, baboon), an ape (e.g., gorilla, chimpanzee, orangutang, gibbon), or a human.

Furthermore, the subject/patient may be any of its forms of development, for example, a foetus.

In one preferred embodiment, the subject/patient is a human.

Suitable subjects for the method may be selected on the basis of conventional factors. Thus, the initial selection of a patient may involve any one or more of: rigorous evaluation by experienced clinician; exclusion of non-AD diagnosis as far as possible by supplementary laboratory and other investigations; objective evaluation of level of cognitive function using neuropathologically validated battery.

In one embodiment, the subject/patient is not a human.

### Methods of Synthesis

Methods for the chemical synthesis of compounds of the present invention are described in the Examples herein. These and/or other well-known methods may be modified and/or adapted in known ways in order to facilitate the synthesis of other compounds of the present invention.

Thus, one aspect of the invention provides a method of synthesising a compound of the invention as described herein, described, or substantially as described, with reference to any of the Examples hereinafter.

The invention further provides a compound of the invention which is obtained by or is obtainable by, a method as described herein.

One aspect of the present invention pertains to methods for the preparation of thiazole-containing TAI compounds, as described herein. The present invention also provides intermediate compounds for use in the preparation of the compounds of the invention.

### General Synthetic Methods

Compounds of formula (I) may be prepared, for example, via routes as set out in the general scheme below:

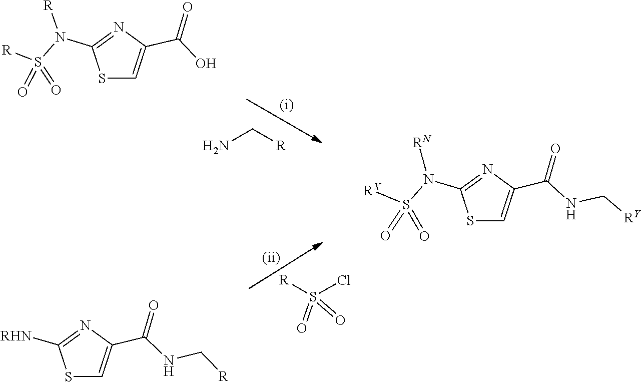

In this scheme, and in all subsequent schemes in this section, substituents may be represented generically as ‘R’. It will be understood that these substituents may independently correspond to groups R^{N}, R^{X} and R^{Y}, or that they may represent protected forms thereof, or precursor groups thereto, as appropriate to the overall synthetic scheme.

For example, route (i) may comprise reacting a sulfonamide thiazole acid with a suitable alkyl amine, in an amide formation step. For example, the acid may be treated with a base (such as diisopropylethylamine DIPEA), a coupling agent (such as HATU), and the amine. Preferably the reaction is carried out in a polar aprotic solvent, such as DMF or MeCN.

For example, route (ii) may comprise reacting an amide thiazolamine with a suitable sulfonyl chloride, for example in the presence of a base (such as triethylamine or DIPEA). Preferably the reaction is carried out in a polar aprotic solvent such as THF.

Starting materials for the synthesis of the compounds of the invention may be prepared via known routes and/or may be commercially available. Methods for the synthesis of exemplary compounds are described in the Examples herein. These and/or other known methods may be modified and/or adapted in known ways, in order to facilitate the synthesis of other compounds of the present invention.

***

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organisational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example+/−10%.

### EXAMPLES

### Example 1

### Modelling of Monomer Capture by PHF Core Oligomer; Immunochemical Analysis of Assembly; Characterisation of HMT-Binding Pocket

As described in detail in WO2022/008545, molecular dynamics simulations were performed to explore the conformational landscape of a dGAE monomer. The inventors further developed a panel of single-chain, monoclonal antibody fragments (scAbs) which recognise linear epitopes spanning most of the core tau unit, and used these to investigate epitope availability in dot immunoblots over the course of dGAE assembly in vitro [6]. They hypothesised that the inhibitory action of HMT on dGAE assembly might be explained by stabilisation of transient cryptic ligand-binding pockets by ligand-induced conformational funnelling [40] and eventually identified a single complex in which HMT remained tightly bound and the protein structure did not vary greatly after 100 ns of MD simulation time. The HMT-bound complex therefore represents an energy well in which the conformation is maintained over a relatively long period. The conformation of the tau297-391 core unit that is stabilised by HMT is a compact folded state which lacks any β-sheets and is very different from the extended conformation required for the monomer to align onto the PHF core template.

### Pharmacophore Modelling of the Cryptic Druggable Pocket and its Use to Identify Novel Inhibitors

The site bound by HMT was determined to be 70% druggable [41], i.e., having a favorable fraction of hydrophobic solvent-accessible surface area. Also as disclosed in WO2022/008545, the inventors developed a pharmacophore model based on the key residues that could be used to assist with the rational design of TAIs. The HMT-binding pocket is predominantly hydrophobic in nature (Phe378, Phe346, Val350, Leu315, Ile354, Ile371). A number of residues have the potential to form hydrogen bonds with a molecule bound within this pocket, including Lys347, Thr373, Leu315 and the NH of Glu372. The inventors exploited the shape, hydrogen-bonding and lipophilic features observed with HMT to design alternative TAIs in the pharmacophore model.

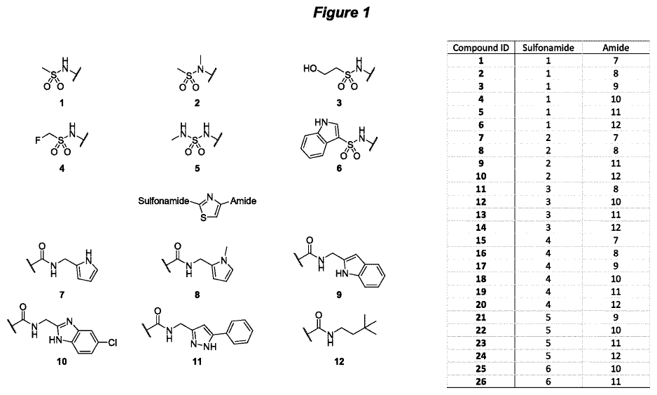

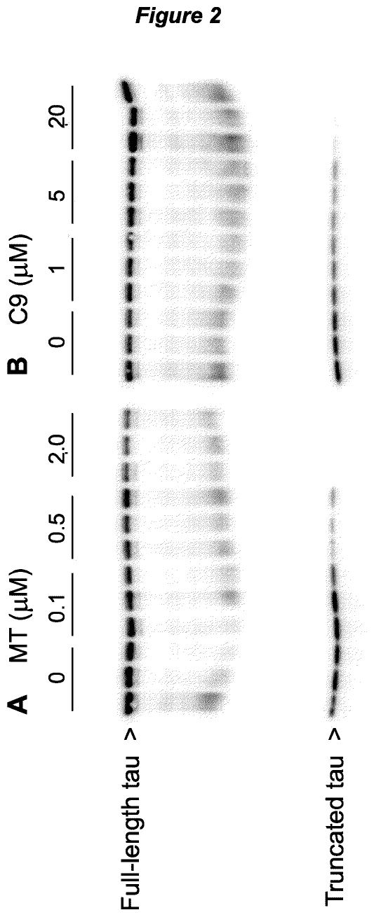

Using computer-aided drug design approaches (see below), the inventors identified a thiazole core as a suitable heterocyclic replacement for the central ring of HMT. A representative range of candidates was synthesised and tested in a cell-based tau aggregation screening assay (see below and FIG. 1), which measures the ability of compounds to inhibit the capture of full-length tau by dGAE and its templated truncation by endogenous proteases [13]. In this model, MT at 1 μM reduces aggregation to 9.6% of that measured in its absence. Of the compounds synthesised and tested, Compound 9 reduced the level of tau aggregation to 26.6% of that measured in the absence of compound. The concentrations required for 50% inhibition (EC₅₀) from a single experiment were 0.6 μM for MT and 3.65 μM for Compound 9 (FIG. 2). Data from replicate experiments are tabulated below in Example 3.

Comparison of the inhibitory activity of the tested thiazole derivatives shows that interactions with Lys343, via hydrogen bonding and π-cation interactions, are important in determining compound potency. The orientation of the sulfonamide oxygen atoms is key to obtaining the desired hydrogen bonding with the backbone carbonyl of Lys343. There is a change in potency when substituting a methyl group onto amide 7 to produce amide 8. This is accounted by a reorientation of the thiazole amide which forms a key H-bond to the backbone NH of Lys347 with compound 2 compared to the compound 1 where this interaction is perturbed by a new interaction with the pyrrole NH with Thr373. A small alkyl substituent appears to be adequate for binding into the lipophilic pocket towards Phe378. The sulfonamide affects the ability of the amide moiety to form a hydrogen bond to the NH of Thr373. Amide substituents 9 and 11 both have the ability to form additional hydrogen bonds with the protein, with the Asp345 backbone carbonyl with 9 or the Leu315 with 11. Compound 9 fulfils a number of the required binding features, including the sulfonamide oxygen hydrogen bonding to the Glu372 backbone NH, the amide carbonyl forming a hydrogen bond to the Lys347 backbone NH and the pyrazole forming a hydrogen bond to the NH of Thr373, and the carbonyl backbone of Leu315. Additionally, the phenyl substituent on the pyrazole neatly binds in the lipophilic pocket, forming a face-edge π stack with Phe378, and is well positioned to interact with Lys343 through π-cation interactions. Therefore, the pharmacophore model developed on the basis of HMT binding permits identification of compounds unrelated chemically to HMT which also have the ability to inhibit tau aggregation.

### Immunochemical Confirmation of TAI Activity

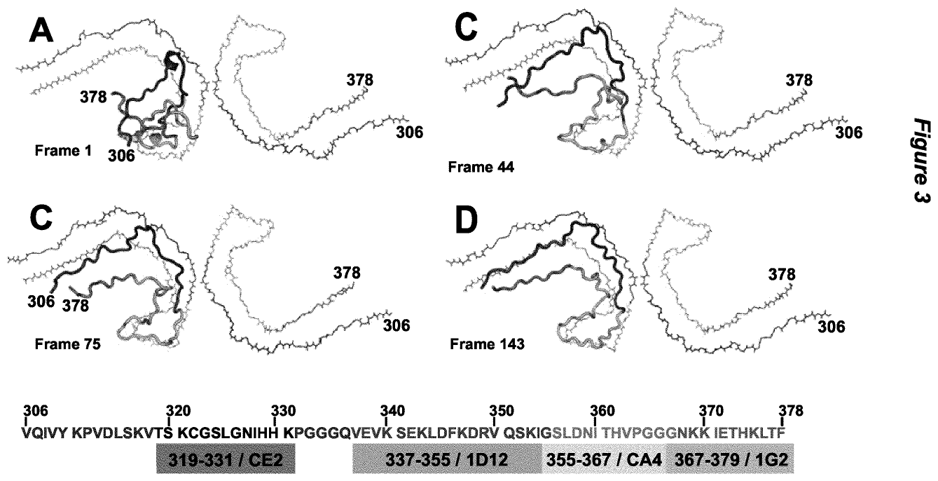

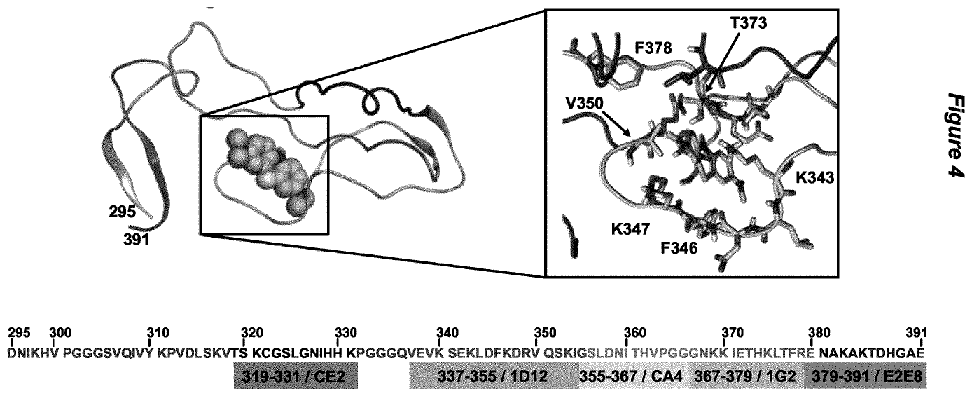

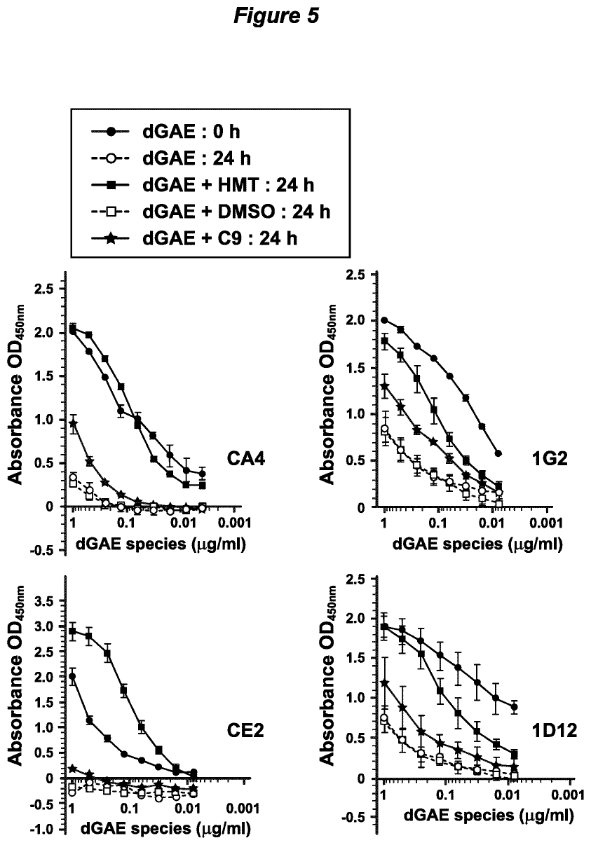

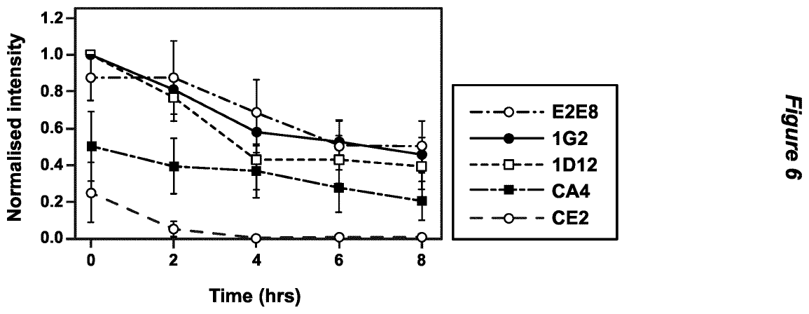

As a separate confirmation of activity, an aqueous phase ELISA assay was used to measure TAI activity in dGAE preparations (see Example 3, below). Immunoreactivity with the antibody panel (whose epitopes are depicted in FIGS. 3 and 4) was measured before and after assembly of dGAE in the presence or absence of HMT and Compound 9. Immunoreactivity over a range of dGAE dilutions indicates that all epitopes were available for antibody binding in the soluble pre-assembly dGAE preparation (FIG. 5). In the assembled dGAE preparation, the CA4 and CE2 epitopes become almost completely unavailable, whereas the availability of the epitopes for 1G2 and 1D12 were substantially reduced, consistent with the immunoblot analyses shown in FIG. 6. When dGAE assembly was performed in the presence of HMT, assembly-dependent occlusion of these epitopes did not occur. The CE2 epitope became more available in the HMT-treated sample than in the soluble pre-assembly sample, suggesting reversal of partial oligomerisation in solution prior to induction of assembly. The 1G2 epitope remained partially occluded in the HMT assembly preparation, consistent with Thr373 (within the 1G2 epitope), being a critical HMT binding site. Epitope exposure was less complete for Compound 9. Although comparable to HMT, as measured by 1G2 immunoreactivity, there was only partial exposure of the CA4 and 1D12 epitopes. Whereas HMT binding prevented the conformational changes in the CE2 epitope responsible for early loss of immunoreactivity, this was not the case for Compound 9, a feature which may account for the lower potency of this compound in the cell assay.

### Computer Aided Drug Design (CADD)

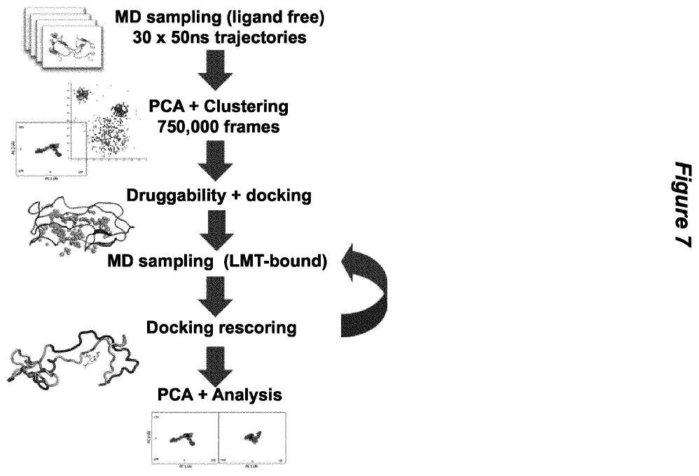

Energy minimisation and 50-ns molecular dynamics simulation on 30 replicas of ligand-free single-stranded filament (FIG. 7). Perform principal component analysis along simulation trajectories identified clusters. Took representative sample protein conformations from each cluster, determined the RMSD between snapshots. Used 380 different protein structures for docking and identified to get 15,090 ligand poses. Scored poses and found 178 binding poses to 65 binding sites. Run a short minimisation and then a 1 ns production run on each system. Recalculate the ligand binding energy. Take the top scoring ligands and extend the simulation times and repeat the process numerous times. Trajectories of interest are processed for analysis.

### Example 2—Chemical Synthesis

All reactions were carried out under positive pressure of argon unless otherwise stated. Glassware was oven-dried at 120° C., or flame-dried under vacuum unless otherwise noted. Anhydrous dichloromethane (DCM), acetonitrile (MeCN), N,N-dimethylformamide (DMF), and pyridine were purchased from Sigma-Aldrich. Anhydrous tetrahydrofuran (THF) was purchased from Acros Organics or Sigma-Aldrich. Other commercially available solvents or reagents were used without further purification unless otherwise noted. Ambient temperature as written is 20-21° C. Reactions were monitored by thin layer chromatography (TLC) using precoated silica gel plates from EMD Millipore (TLC Silica gel 60 F254). Flash column chromatography was performed over Silica gel 60 (particle size 0.04-0.063 mm) from Fluorochem or on a Biotage® Selekt instrument using prepacked columns from Biotage®. ¹H NMR and ¹³C NMR spectra were recorded on a Bruker Avance II 400 MHz & 600 MHz spectrometer at 298 K and the residual solvent peaks were used as internal standards: CDCl₃, 7.26 ppm (¹H) and 77.16 ppm (¹³C); CD₃OD, 3.31 ppm (¹H) and 49.00 ppm (¹³C); DMSO-d₆, 2.50 ppm (¹H) and 39.52 ppm (¹³C). NMR data are denoted with apparent multiplicities: m=multiplet, s=singlet, d=doublet, t=triplet, and combinations thereof; br denotes a broad, poorly resolved signal. Mass spectra were obtained on a Waters Xevo®G2 QTOF mass spectrometer. IR spectra were recorded on a PerkinElmer FT-IR Spectrum Two spectrometer with an ATR Diamond cell. Melting points were obtained using a Stuart® melting point apparatus SMP50 and are uncorrected.

### Synthesis of tert-butyl 4-(3,3-dimethylbutylcarbamoyl)thiazol-2-ylcarbamate

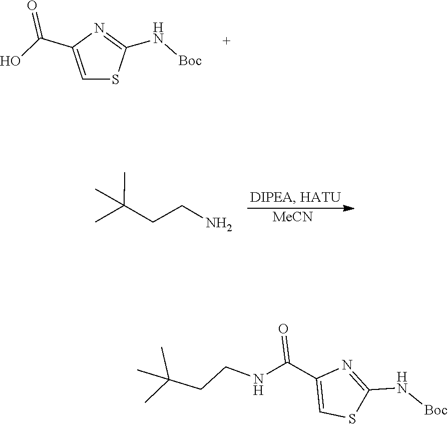

To a stirred suspension of 2-Boc-aminothiazole-4-carboxylic acid (253 mg, 1.04 mmol) in anhydrous MeCN (20 mL) was added DIPEA (269 mg, 362 μL, 2.08 mmol) under an inert atmosphere. Once the solid was fully dissolved HATU (407 mg, 1.07 mmol) was added, followed by 3,3-dimethylbutylamine (108 mg, 144 μL, 1.07 mmol). The reaction mixture was heated at 50° C. for 23 h. Water (40 mL) was added, and the resultant suspension was cooled to ambient temperature. More water (20 mL) was added, and the suspension was vacuum-filtered. The collected solid was washed with water (3×10 mL) and dried in a vacuum oven at 40° C. for 2 h; the title compound (246 mg, 73%) was obtained as an off-white solid.
¹H (400 MHz, CDCl₃) δ 7.73 (br s, 1H), 7.67 (s, 1H), 6.98 (br s, 1H), 3.45-3.39 (m, 2H), 1.55 (s, 11H, includes H₂O signal), 1.53-1.49 (m, 2H), 0.96 (s, 9H);
¹³C (101 MHz, CDCl₃) δ 161.18, 158.86, 151.96, 145.06, 117.18, 83.43, 43.45, 36.12, 30.13, 29.57, 28.29

### Synthesis of 2-amino-N-(3,3-dimethylbutyl)thiazole-4-carboxamide

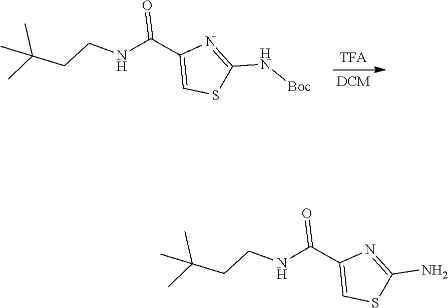 [SMILES: CC(C)(C)CCNC(=O)c1csc(NC(=O)OC(C)(C)C)n1 ; CC(C)(C)CCNC(=O)c1csc(N)n1]

To a stirred solution of tert-butyl 4-(3,3-dimethylbutylcarbamoyl)thiazol-2-ylcarbamate (233 mg, 0.712 mmol) in DCM (6 mL) was added dropwise TFA (2 mL) under an inert atmosphere. The reaction mixture was stirred at 20° C. for 4 h, then concentrated to leave an orange residue, which was dissolved in EtOAc (20 mL). The resultant solution was washed with sat. aq. NaHCO₃ (10 mL) and brine (10 mL). The organic phase was dried (MgSO₄), filtered, and evaporated to leave a viscous, orange oil, which was purified by flash chromatography (SiO₂, EtOAc:petroleum ether 40/60, 7:3); the title compound (171 mg) was obtained as a yellow oil. The material was used without further purification.
¹H (400 MHz, CDCl₃) δ 7.33 (s, 1H), 7.01 (br s, 1H), 4.98 (br s, 2H), 3.42-3.37 (m, 2H), 1.52-1.48 (m, 2H), 0.95 (s, 9H);
¹³C (101 MHz, CDCl₃) δ 168.93, 159.42, 141.20, 112.81, 42.86, 36.70, 30.02, 29.44

### Synthesis of methyl 2-(N-(4-(3,3-dimethylbutylcarbamoyl)thiazol-2-yl)sulfamoyl)acetate

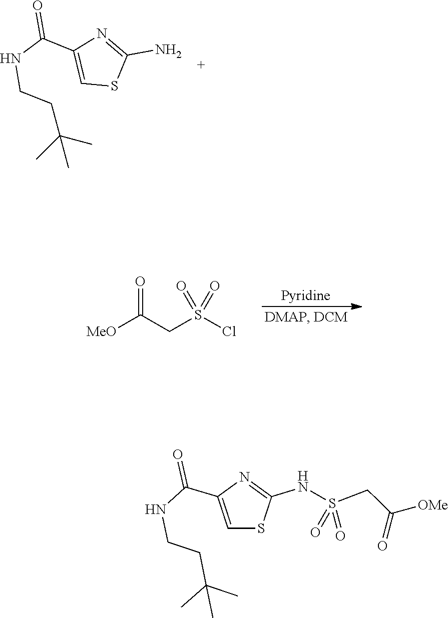 [SMILES: CC(C)(C)CCNC(=O)c1csc(N)n1 ; COC(=O)CS(=O)(=O)Cl ; COC(=O)CS(=O)(=O)Nc1nc(C(=O)NCCC(C)(C)C)cs1]

To a stirred solution of 2-amino-N-(3,3-dimethylbutyl)thiazole-4-carboxamide (123 mg, 0.541 mmol) in DCM (5 mL) was added pyridine (65 mg, 66 μL, 0.816 mmol) and DMAP (14 mg, 0.115 mmol) under an inert atmosphere. Methyl 2-(chlorosulfonyl)acetate (98 mg, 66 μL, 0.568 mmol) was added dropwise, and the resultant solution was stirred at 20° C. for 43 h. The reaction mixture was diluted with DCM (5 mL) and washed with 0.5 M aq. HCl (10 mL), water (10 mL), and brine (10 mL). The brine was extracted with EtOAc (10 mL), and the organic extracts were combined, dried (MgSO₄), filtered, and evaporated to leave yellow and white solid material. The crude product was purified by flash chromatography (SiO₂, DCM:MeOH, 19:1, then 9:1), and the isolated material was further purified by flash chromatography (SiO₂, EtOAc:MeOH, 98:2, then 9:1); the title compound (112 mg) was obtained slightly impure, as a pink-orange foam. The material was used without further purification.
¹H (400 MHz, CD₃OD) δ 7.41 (s, 1H), 4.16 (s, 2H), 3.69 (s, 3H), 3.37-3.33 (m, 3H, includes MeOH), 1.51-1.47 (m, 2H), 0.94 (s, 9H)

### Synthesis of N-(3,3-dimethylbutyl)-2-(2-hydroxyethylsulfonamido)thiazole-4-carboxamide

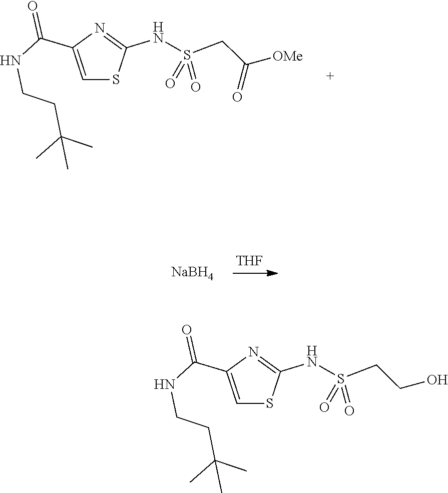 [SMILES: COC(=O)CS(=O)(=O)Nc1nc(C(=O)NCCC(C)(C)C)cs1 ; "NaBH4" ; CC(C)(C)CCNC(=O)c1csc(NS(=O)(=O)CCO)n1 ; "THF"]

To a stirred solution of a crude sample of methyl 2-(N-(4-(3,3-dimethylbutylcarbamoyl)thiazol-2-yl)sulfamoyl)acetate (103 mg, 0.283 mmol) in anhydrous THF (5 mL) was added sodium borohydride (43 mg, 1.14 mmol) under an inert atmosphere. The resultant suspension was stirred at 20° C. for 27 h and quenched with sat. aq. NH₄Cl (20 mL). The mixture was extracted with EtOAc (2×20 mL). The aqueous layer was acidified with 3 drops of 32% aq. HCl, saturated with NaCl, and extracted with DCM (3×15 mL). The DCM was dried (MgSO₄), filtered, and evaporated to leave a white solid. The crude product was dissolved in DCM (15 mL) and extracted with a 3:1 v/v mixture of water and sat. aq. NaHCO₃ (20 mL). The aqueous layer was washed with DCM (15 mL) then acidified to pH ≈1 with 1 M aq. HCl. The acidic solution was extracted with DCM (2×15 mL) and the two combined DCM extracts were washed with brine (10 mL), dried (MgSO₄), filtered, and evaporated to leave a white solid. After drying in a vacuum oven at 40° C. for 1.5 h, the title compound (38 mg, 40%, 23% over 2 steps) was obtained as a white solid.
IR uₘₐₓ (cm⁻¹) 3480, 3313, 3095, 2960, 1645, 1544, 1267, 1239, 1116, 1041, 896, 659, 553, 535;
¹H (400 MHz, DMSO-d₆) δ 12.71 (br s, 1H), 8.43 (br s, 1H), 7.44 (s, 1H), 4.78 (br s, 1H), 3.73 (t, J=6.7 Hz, 2H), 3.24-3.19 (m, 4H), 1.43-1.39 (m 2H), 0.91 (s, 9H);
¹³C (101 MHz, DMSO-d₆) δ 166.90 (br), 157.34, 132.98 (br), 111.50 (br), 55.83, 55.41, 42.51, 35.59, 29.65, 29.22;
HRMS: m/z (ESI) calcd for C₁₂H₂₁N₃NaO₄S₂, 358.0871 (M+Na)⁺; found, 358.0875

### Synthesis of tert-butyl (4-(((1-methyl-1H-pyrrol-2-yl)methyl) carbamoyl)thiazol-2-yl) carbamate

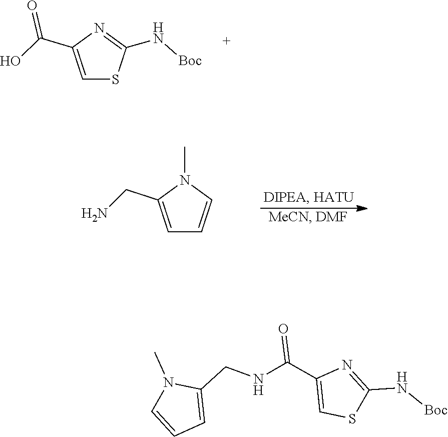

To a stirred solution of 2-Boc-aminothiazole-4-carboxylic acid (526 mg, 2.05 mmol), DIPEA (530 mg, 714 μL, 4.10 mmol) and HATU (779 mg, 2.05 mmol) in a mixture of anhydrous MeCN (15 mL) and DMF (10 mL) under an inert atmosphere was added 1-(1-methyl-1H-pyrrol-2-yl)methanamine (226 mg, 2.05 mmol) and the reaction mixture was stirred at 20° C. for 15 h. The solvents were evaporated, and the residue was dissolved in EtOAc (40 mL) and washed with water (15 mL) and brine (15 mL). The aqueous washes were extracted with EtOAc (2×20 mL), and the combined organic extracts were dried (MgSO₄), filtered and evaporated to give a solid that was purified by flash chromatography (SiO₂, Hexane/EtOAc, 1:1); the title compound (561 mg, 81%) was obtained as a cream solid after drying under high vacuum at 20° C. for 3.5 h.
¹H (400 MHz, CDCl₃) δ 7.73 (br s, 1H), 7.72 (s, 1H), 7.10-7.19 (m, 1H), 6.63 (s, 1H), 6.14 (s, 1H), 6.09 (s, 1H), 4.59 (d, J=5.3 Hz, 2H), 3.60 (s, 3H), 1.55 (s, 9H);
¹³C (101 MHz, CDCl₃) δ 160.55, 158.76, 151.84, 144.41, 128.48, 123.10, 117.50, 108.90, 106.95, 83.29, 34.92, 33.80, 28.11

### Synthesis of 2-amino-N-((1-methyl-1H-pyrrol-2-yl)methyl)thiazole-4-carboxamide

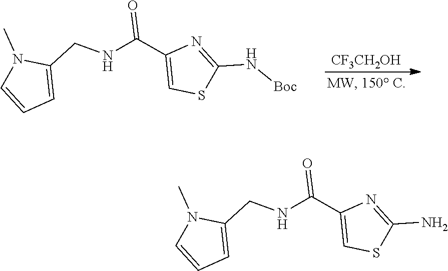 [SMILES: Cn1cccc1CNC(=O)c1csc(NC(=O)OC(C)(C)C)n1 ; "CF3CH2OH" ; Cn1cccc1CNC(=O)c1csc(N)n1]

A solution of tert-butyl (4-(((1-methyl-1H-pyrrol-2-yl)methyl) carbamoyl)thiazol-2-yl) carbamate (265 mg, 0.788 mmol) in 2,2,2-trifluoroethanol (3.5 mL) was placed in a sealed microwave vial and heated to 150° C. with stirring (1000 rpm) for 1.5 h. The solvent was removed by evaporation and the residue was purified by flash chromatography (SiO₂, Hexane/EtOAc, 1:1) to give the title compound (135 mg, 73%) as a white solid after drying at 60° C. for 19 h.
¹H (400 MHz, CDCl₃) δ 7.39 (s, 1H), 7.15 (br s, 1H), 6.62 (s, 1H), 6.13 (s, 1H), 6.08 (s, 1H), 4.88 (br s, 2H), 4.57 (d, J=5.6 Hz, 2H), 3.60 (s, 3H);
¹³C (101 MHz, CDCl₃) δ 166.71, 160.62, 145.50, 128.48, 123.07, 113.85, 108.94, 106.90, 34.91, 33.81

### Synthesis of methyl (4-(((1-methyl-1H-pyrrol-2-yl)methyl) carbamoyl)thiazol-2-yl) sulfonyl)acetate

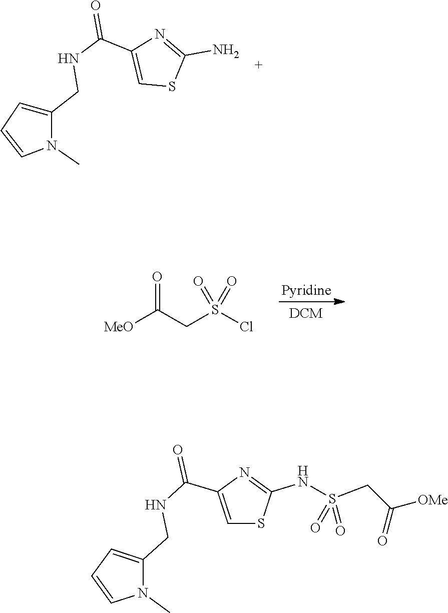 [SMILES: Cn1cccc1CNC(=O)c1csc(N)n1 ; COC(=O)CS(=O)(=O)Cl ; COC(=O)CS(=O)(=O)Nc1nc(C(=O)NCc2cccn2C)cs1]

To a stirred solution of 2-amino-N-((1-methyl-1H-pyrrol-2-yl)methyl)thiazole-4-carboxamide (55 mg, 0.233 mmol) and pyridine (28 mg, 28 μL, 0.349 mmol) in anhydrous DCM (5 mL) under an inert atmosphere was added dropwise a solution of methyl (chlorosulfonyl)acetate (42 mg, 0.245 mmol) in DCM (150 μL), and the resulting solution was stirred at 21° C. for 17 h. The reaction mixture was diluted with DCM (45 mL), washed with 1 M aq. HCl (10 mL), brine (15 mL) and dried (MgSO₄). After filtration the solvent was removed by evaporation to give the title compound (74 mg, 85%) as a tan solid after drying in a vacuum oven at 40° C. for 6.5 h. The material was used without any further purification.
¹H (400 MHz, DMSO-d₆) δ 13.09 (br s, 1H), 8.80 (br s, 1H), 7.58 (s, 1H), 6.68 (s, 1H), 5.98 (s, 1H), 5.90 (s, 1H), 4.38 (d, J=2.7 Hz, 2H), 4.15 (s, 2H), 3.60 (s, 3H), 3.55 (s, 3H);
¹³C (101 MHz, DMSO-d₆) δ 168.74, 164.03, 156.85, 132.27, 128.35, 122.57, 112.42, 108.50, 106.24, 57.35, 52.41, 34.64, 33.37

### Synthesis of 2-(2-hydroxyethylsulfonamido)-N-((1-methyl-1H-pyrrol-2-yl)methyl)thiazole-4-carboxamide

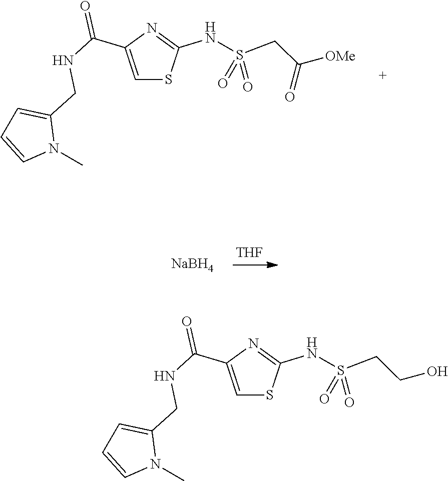 [SMILES: COC(=O)CS(=O)(=O)Nc1nc(C(=O)NCc2cccn2C)cs1 ; "NaBH4" ; Cn1cccc1CNC(=O)c1csc(NS(=O)(=O)CCO)n1 ; "THF"]

To a stirred solution of methyl (4-(((1-methyl-1H-pyrrol-2-yl)methyl) carbamoyl)thiazol-2-yl) sulfonyl)acetate (74 mg, 0.198 mmol) in anhydrous THF (5 ml) was added sodium borohydride (30 mg, 0.792 mmol), and the resulting solution was stirred under an inert atmosphere at 20° C. for 18 h. The solvent was evaporated and the residue was dissolved in water (7 mL), acidified with 1 M aq. HCl (2 mL), saturated with NaCl, and extracted with EtOAc (5×10 mL). The combined organic extracts were washed with brine (15 mL), dried (MgSO₄), filtered and evaporated to give a solid that was dissolved in sat. aq. NaHCO₃ solution (25 mL) and extracted with EtOAc (3×10 mL). The aqueous phase was acidified by dropwise addition of 32% aq. HCl, saturated with NaCl, and extracted with EtOAc (4×25 mL). The combined organic extracts were washed with brine (15 mL), dried (MgSO₄), filtered and evaporated to give a solid that was purified by flash chromatography (SiO₂, EtOAc/MeOH, 10:1) to give the title compound (26 mg, 38%) as a white solid after drying at 60° C. for 3 h.
IR uₘₐₓ (cm⁻¹): 3315, 3097, 2924, 1627, 1554, 1470, 1229, 1102, 713, 504;
¹H (400 MHz, CD₃OD) δ 7.42 (s, 1H), 6.56-6.61 (m, 1H), 6.03 (br s, 1H), 5.91-5.96 (m, 1H), 4.49 (s, 2H), 3.94 (t, J=6.3 Hz, 2H), 3.55 (s, 3H), 3.30 (m, 2H) (signal partially overlaps CD₃OD signal);
¹³C (101 MHz, CD₃OD) δ 173.93, 164.50, 145.35, 129.62, 123.93, 113.63, 110.09, 107.80, 58.13, 55.83, 36.25, 34.14;
HRMS: m/z (ESI) calcd for C₁₂H₁₆N₄NaO₄S₂, (M+Na)⁺; 367.0511; found, 367.0522

### Synthesis of Sulfonamide Thiazole Esters

### Methyl 2-(2-methoxy-2-oxoethylsulfonamido)thiazole-4-carboxylate

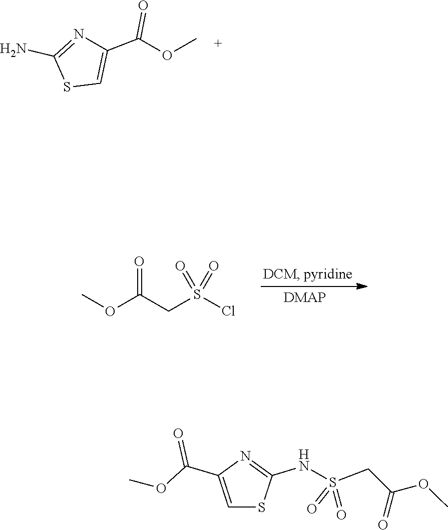

To a stirred suspension of methyl 2-aminothiazole-4-carboxylate (525 mg, 3.15 mmol), pyridine (373 mg, 380 μL, 4.72 mmol) and DMAP (38 mg, 0.315 mmol) in anhydrous DCM (25 mL) was added dropwise methyl (chlorosulfonyl)acetate (571 mg, 3.31 mmol) and the resulting solution was stirred at 20° C. for 17 h. The reaction mixture was diluted with DCM (25 mL) and washed with 1 M aq. HCl (40 mL). The aqueous phase was extracted with DCM (3×50 mL) and the combined organic extracts were washed with brine (40 mL), dried (MgSO₄), filtered and evaporated to give the title compound (920 mg, 99%) as a pale yellow solid. The material was used without further purification.
¹H (400 MHz, DMSO-d₆) δ 13.52 (br s, 1H), 7.76 (s, 1H), 4.20 (s, 2H), 3.82 (s, 3H), 3.61 (s, 3H);
¹³C (101 MHz, DMSO-d₆) δ 168.89, 163.95, 157.88, 128.49, 118.79, 57.40, 52.69, 52.44

### Methyl 2-(2-hydroxyethylsulfonamido)thiazole-4-carboxylate

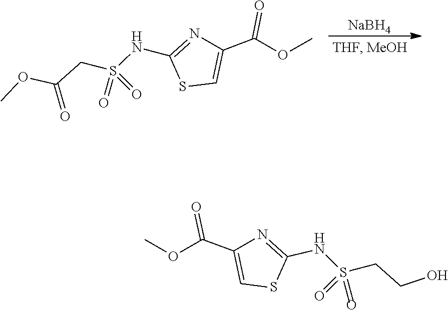

To a stirred solution of 2-(2-methoxy-2-oxoethylsulfonamido)thiazole-4-carboxylate (750 mg, 2.55 mmol) in a mixture of THF (55 mL) and MeOH (6 mL) was added portionwise NaBH₄ (578 mg, 15.29 mmol) over 1.5 h, and the reaction mixture was stirred at 20° C. for 15 h. After this time, NaBH₄ (49 mg, 1.30 mmol) was added and stirring was continued at ambient temperature for 24 h. The reaction mixture was cooled to 0-5° C., brine (25 mL) was added followed by 1 M aq. HCl (22 mL). The organic phase was separated, and the aqueous phase was saturated with NaCl and extracted with THF (5×35 mL). The combined organic extracts were dried (MgSO₄), filtered and evaporated to give a residue that was purified by flash chromatography (SiO₂, DCM/MeOH, 20:1) to give the title compound (403 mg, 59%) as a white solid after drying under high vacuum at 20° C. for 7 h.
¹H (400 MHz, DMSO-d₆) δ 13.09 (br s, 1H), 7.74 (s, 1H), 4.81 (br s, 1H), 3.81 (s, 3H), 3.74 (t, J=6.6 Hz, 2H), 3.23 (m, 2H);
¹³C (101 MHz, DMSO-d₆) δ 166.80, 158.34, 130.04, 118.82, 55.72, 55.45, 52.53

### Methyl 2-(methylsulfonamido)thiazole-4-carboxylate

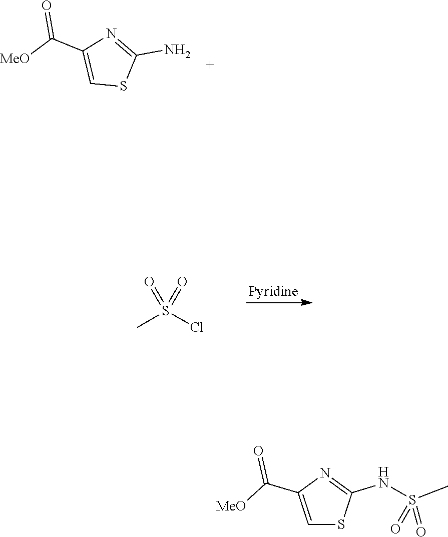

To a stirred solution of methyl 2-aminothiazole-4-carboxylate (801 mg, 5.06 mmol) in anhydrous pyridine (8 mL) was added methanesulfonyl chloride (591 mg, 400 μL, 5.16 mmol) under an inert atmosphere. The reaction mixture was stirred at 20° C. for 20 h then diluted with EtOAc (40 mL). The suspension was filtered, and the filtrate was washed with 1 M aq. HCl (3×40 mL), water (30 mL), and brine (20 mL). The organic phase was dried (MgSO₄), filtered, and evaporated to leave methyl 2-(bis(methylsulfonyl)amino)thiazole-4-carboxylate (190 mg, 23%) as an orange solid.

The aqueous acid was extracted with DCM (70 mL), and the DCM was washed with water (30 mL) and brine (20 mL), then dried (MgSO₄), filtered, and evaporated to leave an orange solid. The combined aqueous extracts were saturated with NaCl, and extracted with DCM (3×30 mL). The combined DCM extracts were washed with brine (20 mL), dried (MgSO₄), filtered, and evaporated to leave a white solid. These two solids obtained from DCM extractions were combined and purified by flash chromatography using a Biotage® Selekt (SiO₂, 5-19% MeOH in DCM); the title compound (586 mg, 49%) was obtained as an off-white solid.
Methyl 2-(bis(methylsulfonyl)amino)thiazole-4-carboxylate: ¹H (400 MHz, DMSO-d₆) δ 8.74 (s, 1H), 3.86 (s, 3H), 3.69 (s, 6H);
¹³C NMR (101 MHz, DMSO-d₆) δ 160.45, 154.18, 143.28, 134.21, 52.43, 43.11
Methyl 2-(methylsulfonamido)thiazole-4-carboxylate: ¹H (400 MHz, CDCl₃) δ 7.31 (s, 1H), 3.92 (s, 3H), 3.06 (s, 3H);
¹³C (101 MHz, CDCl₃) δ 166.71, 158.04, 128.58, 116.55, 53.25, 41.67

### Methyl 2-(fluoromethylsulfonamido)thiazole-4-carboxylate

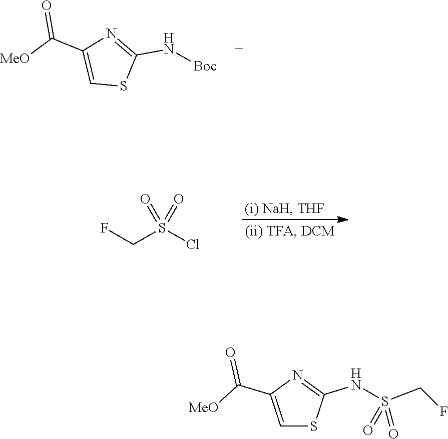 [SMILES: COC(=O)c1csc(NC(=O)OC(C)(C)C)n1 ; O=S(=O)(Cl)CF ; COC(=O)c1csc(NS(=O)(=O)CF)n1]

To a stirred solution of methyl 2-Boc-aminothiazole-4-carboxylate (762 mg, 2.95 mmol) in anhydrous THF (24 ml) under an inert atmosphere was added sodium hydride (60% dispersion in mineral oil, 161 mg, 4.01 mmol). After effervescence had ceased, fluoromethanesulfonyl chloride (351 mg, 2.65 mmol) was added dropwise over 5 min and the reaction mixture was stirred at 20° C. for 70 h. The solvent was evaporated and the residue was dissolved in DCM (60 mL). The reaction mixture was cooled to 0-5° C., TFA (8 mL) was added dropwise, and the resulting solution was stirred at ambient temperature for 22 h. The solvent was removed by evaporation and to the residue was added sat. aq. NaHCO₃ solution (50 mL). The mixture was sonicated for 5 min, vacuum-filtered, and the filtrate was extracted with DCM (10×50 mL). The aqueous phase was acidified by the dropwise addition of 32% aq. HCl and extracted with EtOAc (4×40 mL). The combined organic extracts were washed with brine (40 mL), dried (MgSO₄), filtered and evaporated to give the title compound (365 mg, 54%) as a white solid after drying in an oven at 60° C. for 2 h.
¹H (400 MHz, DMSO-d₆) δ 13.74 (br s, 1H), 7.78 (s, 1H), 5.33 (d, J=46 Hz, 2H), 3.83 (s, 3H);
¹³C (101 MHz, DMSO-d₆) δ 169.70, 157.76, 128.38, 118.96, 90.73 (d, J=208 Hz), 52.70

### Synthesis of methyl 2-(methylsulfamoyl)aminothiazole-4-carboxylate

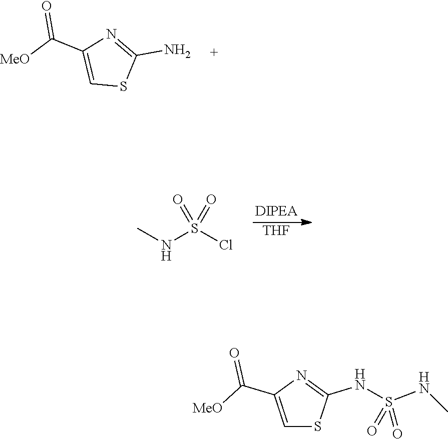

To a stirred solution of methyl 2-aminothiazole-4-carboxylate (499 mg, 3.15 mmol) in anhydrous THF (40 mL) was added under an inert atmosphere DIPEA (449 mg, 605 μL, 3.47 mmol). N-methylsulfamoyl chloride (446 mg, 302 μL, 3.44 mmol) was added dropwise and the resultant solution was heated at 50° C. for 116 h. After cooling to ambient temperature, the THF was evaporated and the residue was partitioned between EtOAc (20 mL) and sat. aq. NaHCO₃ (20 mL). After phase separation, the organic phase was extracted with sat. aq. NaHCO₃ (10 mL) and the combined aqueous phases were washed with EtOAc (20 mL). The aqueous portion was acidified to pH ≈1 with 2 M aq. HCl, extracted with DCM (3×20 mL), saturated with NaCl, and extracted with THF (3×20 mL). The combined organic extracts were dried (MgSO₄), filtered, and evaporated to leave a viscous, orange oil. The crude material was purified by flash chromatography (SiO₂, DCM:MeOH, 94:6), and the title compound (551 mg, 70%) was isolated as a viscous, pale yellow oil.
¹H (400 MHz, CD₃OD) δ 7.64 (s, 1H), 3.89 (s, 3H), 2.62 (s, 3H);
¹³C (101 MHz, CD₃OD) δ 167.27, 160.63, 133.61, 119.64, 53.09, 29.47

### Synthesis of methyl 2-(1H-indole-3-sulfonamido)thiazole-4-carboxylate

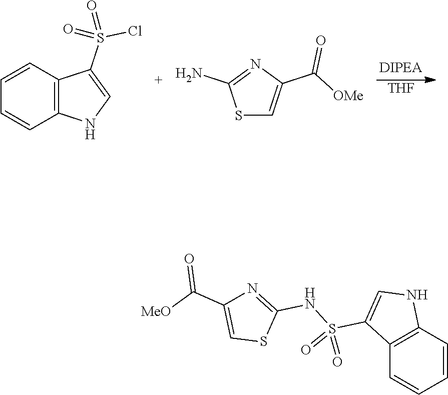 [SMILES: O=S(=O)(Cl)c1c[nH]c2ccccc12 ; COC(=O)c1csc(N)n1 ; COC(=O)c1csc(NS(=O)(=O)c2c[nH]c3ccccc23)n1 ; "THF"]

To a stirred solution of ¹H-indole-3-sulfonyl chloride (471 mg, 2.18 mmol) in anhydrous THF (20 mL) was added methyl 2-amino-1,3-thiazole-4-carboxylate (693 mg, 4.38 mmol) under an inert atmosphere. DIPEA (312 mg, 420 μL, 2.41 mmol) was added and the resultant solution was heated at 50° C. for 116 h. After cooling to ambient temperature, the THF was evaporated and the residue was partitioned between EtOAc (40 mL) and sat. aq. NaHCO₃ (20 mL). After phase separation, the organic phase was extracted with sat. aq. NaHCO₃ (20 mL), and the combined aqueous extracts were washed with EtOAc (20 mL). The aqueous phase was acidified to pH ≈1 with 2 M aq. HCl, and a sticky orange solid appeared, stuck to the glass vessel. The mixture was extracted with DCM (20 mL), which did not dissolve the sticky solid. Nonetheless, the DCM was dried (MgSO₄), filtered, and evaporated to leave a viscous, yellow oil. The oil was dissolved in a mixture of DCM and MeOH and concentrated to leave the title compound (265 mg, 36%) as a pale yellow foamy solid.

The aqueous phase was saturated with NaCl, and extracted with THF (4×20 mL). The combined THF extracts were dried (MgSO₄), filtered, and evaporated to leave a pink-orange foam, which was purified by flash chromatography using a Biotage® Selekt (SiO₂, 4-20% MeOH in DCM). Additional title compound (138 mg, 19%) was isolated as a pale yellow solid. Total yield; 403 mg (55%).
¹H NMR (400 MHz, CD₃OD) δ 7.87 (s, 1H), 7.84 (d, J=7.7 Hz, 1H), 7.44-7.41 (m, 2H), 7.22-7.17 (m, 1H), 7.16-7.12 (m, 1H), 3.79 (s, 3H);
¹³C (101 MHz, CD₃OD) δ 168.17, 159.87, 137.95, 131.89, 130.64, 124.74, 124.25, 122.42, 120.72, 118.97, 115.97, 113.20, 53.04

### Synthesis of methyl 2-(N,S-dimethylsulfonamido)thiazole-4-carboxylate

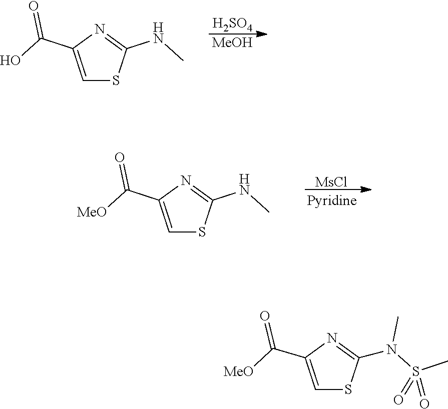

To a stirred suspension of 2-(methylamino)thiazole-4-carboxylic acid (501 mg, 3.17 mmol) in MeOH (12 mL) was added concentrated sulfuric acid (1.0 mL) under an inert atmosphere. The resultant solution was heated at reflux for 15 h. After cooling to ambient temperature, the MeOH was evaporated and the residue was diluted with DCM (20 mL) and water (10 mL). The aqueous phase was neutralised with sat. aq. NaHCO₃, and the phases were separated. The aqueous phase was extracted with DCM (15 mL), and the combined organic phases were washed with water (15 mL) and brine (15 mL). The DCM was dried (MgSO₄), filtered, and evaporated to leave methyl 2-(methylamino)thiazole-4-carboxylate (430 mg, 79%) as an off-white solid.
¹H (400 MHz, CDCl₃) δ 8.05 (s, 1H), 7.34 (s, 1H), 3.85 (s, 3H), 3.00 (s, 3H);
¹³C (101 MHz, CDCl₃) δ 172.07, 162.16, 143.15, 115.00, 52.05, 32.65

To a stirred suspension of methyl 2-(methylamino)thiazole-4-carboxylate (200 mg, 1.16 mmol) in anhydrous pyridine (4 mL) was added methanesulfonyl chloride (266 mg, 180 μL, 2.32 mmol) under an inert atmosphere. The reaction mixture was stirred at 20° C. for 21 h then diluted with DCM (20 mL). The mixture was washed with 1 M aq. HCl (3×20 mL), water (20 mL), and brine (10 mL). The organic phase was dried (MgSO₄), filtered, and evaporated to leave the title compound (227 mg, 78%) as an orange solid.
¹H (400 MHz, CDCl₃) δ 7.84 (s, 1H), 3.91 (s, 3H), 3.58 (s, 3H), 3.03 (s, 3H);
¹³C (101 MHz, CDCl₃) δ 161.70, 161.65, 142.78, 124.53, 52.57, 37.60, 36.85

### Synthesis of Sulfonamido Thiazole Acids: General Procedure A

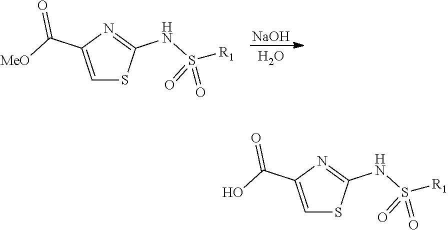

To a sulfonamido thiazole ester (1 eq.) was added an aqueous solution of NaOH (3-6 eq.), and the resultant mixture was stirred at ambient temperature for 3-6 h. The reaction mixture was filtered, the filtrate was washed with EtOAc (15 mL), and the aqueous layer was acidified with 1 M aq. HCl to pH ≈1. The acidic solution was saturated with NaCl then extracted with DCM (3×10 mL) and THF (3×15 mL). The combined THF extracts were washed with brine (10 mL), dried (MgSO₄), filtered, and evaporated.

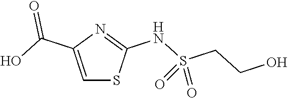 [SMILES: O=C(O)c1csc(NS(=O)(=O)CCO)n1]

Following General Procedure A using methyl 2-(2-hydroxyethylsulfonamido)thiazole-4-carboxylate (500 mg, 1.88 mmol) and 1 M NaOH (3.2 eq.) afforded 2-(2-hydroxyethylsulfonamido)thiazole-4-carboxylic acid as a white solid (426 mg, 90%).
¹H (400 MHz, DMSO-d₆) δ 12.71-14.16 (br s, 3H), 7.61 (s, 1H), 3.73 (t, J=6.8 Hz, 2H), 3.19 (t, J=6.6 Hz, 2H);
¹³C (101 MHz, DMSO-d₆) δ 167.43, 159.01, 130.05, 117.55, 55.78, 55.46

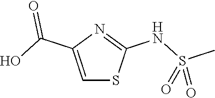

Following General Procedure A, using methyl 2-(methylsulfonamido)thiazole-4-carboxylate (626 mg, 2.65 mmol) and 1 M NaOH (3.3 eq.) afforded 2-(methylsulfonamido)thiazole-4-carboxylic acid as a pale yellow solid (473 mg, 80%).
¹H (400 MHz, CD₃OD) δ 7.52 (s, 1H), 2.99 (s, 3H);
¹³C (101 MHz, CD₃OD) δ 169.48, 160.24, 131.36, 118.13, 41.15

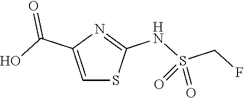 [SMILES: O=C(O)c1csc(NS(=O)(=O)CF)n1]

Following General Procedure A using methyl 2-(fluoromethylsulfonamido)thiazole-4-carboxylate (362 mg, 1.42 mmol) and 1 M NaOH (5.6 eq.) afforded 2-(fluoromethylsulfonamido)thiazole-4-carboxylic acid as an off-white solid (317 mg, 93%).
¹H (400 MHz, DMSO-d₆) δ 14.70-11.95 (br s, 2H), 7.67 (s, 1H), 5.31 (d, J=46 Hz, 2H);
¹³C (101 MHz, DMSO-de) δ 169.90, 158.73, 129.63, 118.06, 90.74 (d, J=208 Hz)

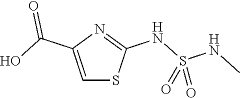

Following General Procedure A, using methyl 2-(methylsulfamoyl)aminothiazole-4-carboxylate (170 mg, 0.677 mmol) and 0.5 M NaOH (5.6 eq.) afforded 2-((N-methylsulfamoyl)amino)thiazole-4-carboxylic acid as a white solid (134 mg, 83%).
¹H (400 MHz, CD₃OD) δ 7.57 (s, 1H), 2.62 (s, 3H);
¹³C (101 MHz, CD₃OD) δ 167.70, 161.31, 134.14, 118.90, 29.48

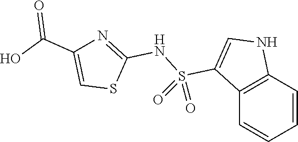 [SMILES: O=C(O)c1csc(NS(=O)(=O)c2c[nH]c3ccccc23)n1]

Following General Procedure A, using methyl 2-(1H-indole-3-sulfonamido)thiazole-4-carboxylate (399 mg, 1.18 mmol) and 0.5 M NaOH (3.2 eq.) afforded 2-(1H-indole-3-sulfonamido)thiazole-4-carboxylic acid as an off-white solid (325 mg, 85%).
¹H (400 MHz, DMSO-d₆) δ 13.21 (br s, 2H), 11.90 (br s, 1H), 7.94 (s, 1H), 7.74 (d, J=7.8 Hz, 1H), 7.61 (s, 1H), 7.47 (d, J=8.0 Hz, 1H), 7.22 (app t, J=7.5 Hz, 1H), 7.16 (app t, J=7.4 Hz, ¹H);
¹³C (101 MHz, DMSO-d₆) δ 159.20 (br), 136.17, 129.54 (br), 123.20, 122.77, 120.99, 119.43, 117.70 (br), 115.05 (br), 112.49 (2 missing)

### Synthesis of 2-(N,S-dimethylsulfonamido)thiazole-4-carboxylic acid

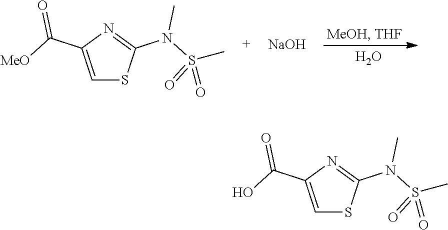

To a solution of methyl 2-(N,S-dimethylsulfonamido)thiazole-4-carboxylate (225 mg, 0.899 mmol) in a mixture of MeOH (6 mL) and THF (3 mL) was added 1 M aq. NaOH (1 mL) under an inert atmosphere. The resultant solution was stirred at 20° C. for 2 h then concentrated. The residue was diluted with water (10 mL) and extracted with DCM (2×10 mL). The aqueous layer was acidified with 1 M aq. HCl (2 mL) and stirred until a precipitate appeared. The resultant suspension was vacuum-filtered, and the collected solid was washed with water (3×10 mL) and dried in a vacuum oven at 40° C. for 13.5 h; the title compound (174 mg, 82%) was obtained as a pale yellow solid.
¹H (400 MHz, CD₃OD/CDCl₃) δ 7.95 (s, 1H), 3.54 (s, 3H), 3.11 (s, 3H);
¹³C (101 MHz, CD₃OD/CDCl₃) δ 163.85, 162.81, 143.97, 125.36, 37.41, 36.95

### Synthesis of Test Inhibitors: General Procedure B

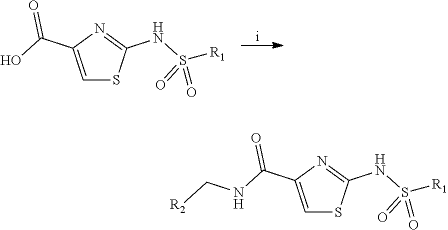

### i; Amine, DIPEA, HATU, MeCN or DMF, 20 or 50° C.

To a stirred suspension of a sulfonamido thiazole acid (1.0 eq.) in anhydrous MeCN or DMF was added DIPEA (2.0 eq.) under an inert atmosphere. Once the solid was fully dissolved, HATU (1.0 eq.) was added, followed by an amine (1.2 eq.). The reaction mixture was stirred at 20° C. or 50° C. N.B. If an amine hydrochloride was used, additional DIPEA was added equivalent to the moles of hydrochloride. Work-up procedures varied and are detailed for each compound.

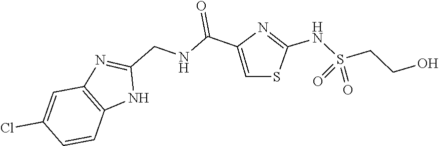 [SMILES: O=C(NCc1nc2cc(Cl)ccc2[nH]1)c1csc(NS(=O)(=O)CCO)n1]

Following General Procedure B, using 2-(2-hydroxyethylsulfonamido)thiazole-4-carboxylic acid (87 mg, 0.345 mmol) and 1-(5 (6)-chloro-1H-benzo[d]imidazol-2-yl)methanamine dihydrochloride (96 mg, 0.379 mmol) in anhydrous MeCN (10 mL) and DMF (1 mL) at 21° C. for 22 h. Water (15 mL) was added and the solution was saturated with NaCl then extracted with EtOAc (4×50 mL). The combined organic extracts were dried (MgSO₄), filtered and evaporated to give a pale yellow oil that was dissolved in sat. aq.

NaHCO₃ solution (25 mL) and extracted with DCM (6×40 mL). The aqueous phase was neutralised by dropwise addition of 32% aq. HCl, saturated with NaCl, and extracted with THF (3×40 mL). The combined organic extracts were dried (MgSO₄), filtered and evaporated to give a solid that was purified by a further two rounds of the base dissolution procedure to give N-((5(6)-chloro-1H-benzo[d]imidazol-2-yl)methyl)-2-(2-hydroxyethylsulfonamido)thiazole-4-carboxamide (118 mg, 82%) as an off-white solid after drying in an oven at 60° C. for 17 h.
IR uₘₐₓ (cm⁻¹) 3404, 3105, 1626, 1548, 1471, 1418, 1255, 1111, 1060, 1016, 841, 727, 559;
¹H (400 MHz, DMSO-d₆) δ 11.94-12.88 (br s, 2H), 8.85 (s, 1H), 7.56 (s, 1H), 7.50 (d, J=8.0 Hz, 1H), 7.40 (s, 1H), 7.16 (d, J=8.6 Hz, 1H), 4.45-4.79 (br s, 1H), 4.62 (d, J=5.3 Hz, 2H), 3.71 (t, J=6.8 Hz, 2H), 3.12 (t, J=6.8 Hz, 2H);
¹³C (101 MHz, DMSO-d₆) δ 168.56, 161.01, 153.87, 140.85, 137.07 (br), 125.86, 121.75, 115.80 (br), 114.86 (br), 112.60, 56.49, 54.45, 37.31 (2 missing);
HRMS: m/z (ESI) calcd for C₁₄H₁₅ClN₅O₄S₂, 416.0250 (M+H)⁺; found, 416.0254

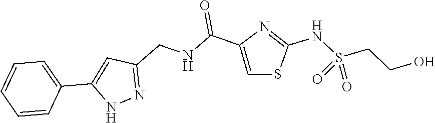 [SMILES: O=C(NCc1cc(-c2ccccc2)[nH]n1)c1csc(NS(=O)(=O)CCO)n1]

Following General Procedure B, using 2-(2-hydroxyethylsulfonamido)thiazole-4-carboxylic acid (86 mg, 0.341 mmol) and (5-phenyl-1H-pyrazol-3-yl)methanamine (65 mg, 0.375 mmol) in anhydrous MeCN (11 mL) and DMF (0.5 mL) at 21° C. for 18 h. Brine (25 mL) was added and the reaction mixture was extracted with THF (4×50 mL). The combined organic extracts were dried (MgSO₄), filtered and evaporated to give a pale yellow oil that was dissolved in sat. aq. NaHCO₃ solution (25 mL) and extracted with DCM (10×50 mL). The aqueous phase was neutralised by dropwise addition of 32% aq. HCl, saturated with NaCl, and extracted with THF (3×25 mL). The combined organic extracts were dried (MgSO₄), filtered and evaporated to give a solid that was suspended in DCM (15 mL) and sonicated for 10 min. The solid was collected by vacuum filtration to give 2-(2-hydroxyethylsulfonamido)-N-((5-phenyl-¹H-pyrazol-3-yl)methyl)thiazole-4-carboxamide (92 mg, 66%) as a white solid after drying in an oven at 60° C. for 19 h.
IR uₘₐₓ (cm⁻¹) 3320, 3209, 1642, 1552, 1303, 1115, 1005, 890, 764, 751, 727, 660, 549;
¹H (400 MHz, DMSO-d₆) δ 12.61-13.20 (br s, 2H), 8.95 (br s, 1H), 7.74 (d, J=7.6 Hz, 2H), 7.55 (s, 1H), 7.41 (app t, J=7.6 Hz, 2H), 7.30 (t, J=7.4 Hz, 1H), 6.59 (s, 1H), 4.79 (br s, 1H), 4.44 (d, J=4.9 Hz, 2H), 3.73 (t, J=6.4 Hz, 2H), 3.21 (m, 2H);
¹³C (101 MHz, DMSO-d₆) δ 166.75, 157.74, 146.04, 134.01, 131.15, 128.82, 127.71, 125.02, 112.36, 101.00, 55.84, 55.35, 35.66 (1 missing);
HRMS: m/z (ESI) calcd for C₁₆H₁₇N₅NaO₄S₂, 430.0617 (M+Na)⁺; found, 430.0620

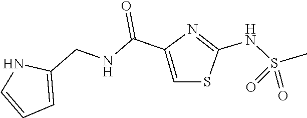 [SMILES: CS(=O)(=O)Nc1nc(C(=O)NCc2ccc[nH]2)cs1]

Following General Procedure B, using 2-(methylsulfonamido)thiazole-4-carboxylic acid (99 mg, 0.445 mmol) and 1-(1H-pyrrol-2-yl)methanamine (47 mg, 0.490 mmol) in anhydrous MeCN (13 mL) at 20° C. for 23 h. Water (30 mL) was added, and the solution was saturated with NaCl then extracted with EtOAc (4×40 mL). The combined organic extracts were washed with brine (20 mL), dried (MgSO₄), filtered and evaporated to give a pale yellow oil that was dissolved in sat. aq. NaHCO₃ solution (30 mL) and extracted with EtOAc (4×20 mL). The aqueous phase was acidified by dropwise addition of 32% aq. HCl and extracted with EtOAc (4×40 mL). The combined organic extracts were washed with brine (30 mL), dried (MgSO₄), filtered and evaporated to give a tan solid that was redissolved in sat. aq. NaHCO₃ solution (35 mL) and extracted with EtOAc (4×20 mL). The aqueous phase was acidified by dropwise addition of 32% aq. HCl and extracted with EtOAc (4×40 mL). The combined organics were washed with brine (30 mL), dried (MgSO₄), filtered and evaporated to give 2-(methylsulfonamido)-N-((1H-pyrrol-2-yl)methyl)thiazole-4-carboxamide as a tan solid (87 mg, 65%) after drying in a vacuum oven at 40° C. for 37 h.
IR uₘₐₓ (cm⁻¹) 3434, 3345, 3142, 1637, 1543, 1512, 1265, 1120, 1107, 738, 520;
¹H (400 MHz, DMSO-d₆) δ 12.74 (br s, 1H), 10.65 (s, 1H), 8.74 (br s, 1H), 7.56 (s, 1H), 6.62-6.69 (m, 1H), 5.89-5.97 (m, 2H), 4.36 (d, J=5.7 Hz, 2H), 2.97 (br s, 3H);
¹³C (101 MHz, DMSO-d₆) δ 166.28, 157.61, 128.03, 117.37, 112.62, 107.40, 106.29, 40.84, 36.02; (1 missing);
HRMS: m/z (ESI) calcd for C₁₀H₁₂N₄NaO₃S₂, 323.0249 (M+Na)⁺; found, 323.0275

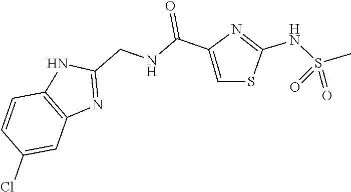 [SMILES: CS(=O)(=O)Nc1nc(C(=O)NCc2nc3cc(Cl)ccc3[nH]2)cs1]

Following General Procedure B, using 2-(methylsulfonamido)thiazole-4-carboxylic acid (80 mg, 0.360 mmol) and 1-(5 (6)-chloro-1H-benzo[d]imidazol-2-yl)methanamine dihydrochloride (90 mg, 0.355 mmol) in anhydrous DMF (0.5 mL) and MeCN (10 mL) at 20° C. for 89 h. Water (25 mL) was added and the solution was saturated with NaCl then extracted with EtOAc (4×30 mL). The combined organic extracts were dried (MgSO₄), filtered and evaporated to give a pale yellow solid that was dissolved in sat. aq. NaHCO₃ solution (25 mL) and extracted with DCM (8×35 mL). The aqueous phase was neutralised by dropwise addition of 32% aq. HCl and extracted with THF (3×25 mL). The combined organic extracts were dried (MgSO₄), filtered and evaporated to give N-((5 (6)-chloro-1H-benzo[d]imidazol-2-yl)methyl)-2-(methylsulfonamido)thiazole-4-carboxamide as an off-white solid (139 mg, “100%”) after trituration with DCM/MeOH and drying at 50° C. under vacuum for 7 h. Traces of solvent (DCM, MeOH and THF) were not diminished on further drying.
IR uₘₐₓ (cm⁻¹) 3631, 3409, 3111, 2988, 1625, 1547, 1471, 1252, 1113, 839, 558;
¹H (400 MHz, DMSO-d₆) δ 12.42 (br s, 2H), 8.73 (br s, 1H), 7.55 (s, 1H), 7.50 (d, J=8.5 Hz, 1H), 7.33 (s, 1H), 7.16 (dd, J=1.2, 8.5 Hz, 1H), 4.63 (d, J=5.9 Hz, 2H), 2.80 (s, 3H);
¹³C (101 MHz, DMSO-d₆) δ 168.54, 160.91, 153.84, 140.72, 137.26 (br), 125.86, 121.76, 115.75 (br), 114.83 (br), 112.58, 39.99, 37.32 (1 missing);
HRMS: m/z (ESI) calcd for C₁₃H₁₂ClN₅O₃S₂, 386.0146 (M+H)⁺; found, 386.0148

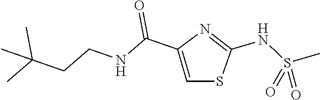 [SMILES: CC(C)(C)CCNC(=O)c1csc(NS(C)(=O)=O)n1]

Following General Procedure B, using 2-(methylsulfonamido)thiazole-4-carboxylic acid (129 mg, 0.580 mmol) and 3,3-dimethylbutylamine (71 mg, 94 μL, 0.699 mmol) in anhydrous MeCN (10 mL) at 20° C. for 93 h. Water (80 mL) was added and the resultant mixture was extracted with DCM (3×30 mL). The combined organic extracts were washed with 1 M aq. HCl (20 mL), water (20 mL), and brine (20 mL). The organic phase was dried (MgSO₄), filtered, and evaporated to leave a viscous, orange oil. The isolated material was partitioned between EtOAc (40 mL) and 1 M aq. HCl (25 mL), and the organic phase was separated and extracted with NaHCO₃ (2×20 mL). The combined basic extracts were acidified with 32% aq. HCl to pH ≈1 and extracted with DCM (2×15 mL). The combined organic extracts were washed with water (15 mL) and brine (10 mL), dried (MgSO₄), filtered, and evaporated to leave N-(3,3-dimethylbutyl)-2-(methylsulfonamido)thiazole-4-carboxamide as a white solid (109 mg, 61%).
IR uₘₐₓ (cm⁻¹) 3335, 3104, 2955, 1645, 1530, 1282, 1125, 970, 886, 652, 529, 518;
¹H (400 MHz, DMSO-d₆) δ 12.65 (br s, 1H), 8.42 (br s, 1H), 7.45 (s, 1H), 3.24-3.19 (m, 2H), 2.98 (br s, 3H), 1.43-1.39 (m, 2H), 0.91 (s, 9H);
¹³C (101 MHz, DMSO-d₆) δ 166.59 (br), 157.43, 133.74 (br), 111.56 (br), 42.52, 40.81, 35.55, 29.64, 29.21;
HRMS: m/z (ESI) calcd for C₁₁H₁₉N₃NaO₃S₂, 328.0766 (M+Na)⁺; found, 328.0772

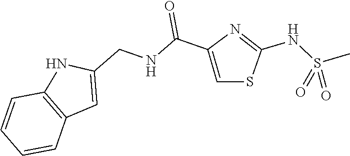 [SMILES: CS(=O)(=O)Nc1nc(C(=O)NCc2cc3ccccc3[nH]2)cs1]

Following General Procedure B, using 2-(methylsulfonamido)thiazole-4-carboxylic acid (98 mg, 0.441 mmol) and (1H-indol-2-yl)methanamine hydrochloride (96 mg, 0.526 mmol) in anhydrous MeCN (5 mL) at 50° C. for 15 h. After cooling to ambient temperature, water (25 mL) was added and the resultant suspension was cooled in an ice bath for 30 min. The liquid was decanted, and the solid residue was dissolved in MeOH (10 mL). The MeOH was evaporated, and the orange residue was dried in a vacuum oven at 40° C. for 80 min. The dried crude material was dissolved in EtOAc (20 mL), and the solution was extracted with 1 M aq. HCl (20 mL) and sat. aq. NaHCO₃ (10 mL). The combined aqueous extracts contained a precipitate, which was collected by vacuum-filtration. The solid was washed with water (3×10 mL) and dried in a vacuum oven at 40° C. for 16.5 h to afford N-((1H-indol-2-yl)methyl)-2-(methylsulfonamido)thiazole-4-carboxamide as a tan solid (47 mg, 30%).
IR uₘₐₓ (cm⁻¹) 2284, 2242, 3099, 1652, 1557, 1533, 1251, 1116, 1103, 789, 734, 645, 540, 513;
¹H (600 MHz, DMSO-d₆) δ 12.75 (br s, 1H), 10.97 (s, 1H), 8.97 (br s, 1H), 7.60 (br s, 1H), 7.45 (d, J=7.8 Hz, 1H), 7.33 (d, J=8.1 Hz, 1H), 7.05-7.02 (m, 1H), 6.96-6.94 (m, 1H), 6.30 (s, 1H), 4.58 (d, J=5.6 Hz, 2H), 2.99 (br s, 3H);
¹³C (151 MHz, DMSO-d₆) δ167.04 (br), 157.76 (br), 136.31, 136.11, 127.87, 120.74, 119.57, 118.88, 112.11 (br), 111.09, 99.32, 40.80, 36.45 (1 missing);
HRMS: m/z (ESI) calcd for C₁₄H₁₄N₄NaO₃S₂, 373.0405 (M+Na)⁺; found, 373.0408

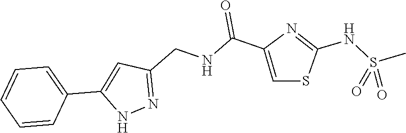 [SMILES: CS(=O)(=O)Nc1nc(C(=O)NCc2cc(-c3ccccc3)[nH]n2)cs1]

Following General Procedure B, using 2-(methylsulfonamido)thiazole-4-carboxylic acid (92 mg, 0.414 mmol) and (5-phenyl-1H-pyrazol-3-yl)methanamine (85 mg, 0.491 mmol) in anhydrous MeCN (10 mL) at 50° C. for 40 h. After cooling to ambient temperature, water (40 mL) was added and the resultant suspension was cooled in an ice bath for 30 min. The solid was collected by vacuum-filtration, washed with water (3×10 mL), and dried in a vacuum oven at 40° C. for 45 min. To remove an aliphatic impurity, the dried solid was partitioned between MeOH (60 mL) and hexane (20 mL), and the resultant suspension was vacuum-filtered. The collected solid was washed with hexane (10 mL) and MeOH (10 mL), and dried in a vacuum oven at 40° C. for 1 h; 2-(methylsulfonamido)-N-((5-phenyl-1H-pyrazol-3-yl)methyl)thiazole-4-carboxamide was obtained as a pale tan solid (77 mg, 49%). The two phases evident in the filtrate were separated, the MeOH phase was concentrated, and the residue was dried in a vacuum oven at 40° C. for 4 h; additional 2-(methylsulfonamido)-N-((5-phenyl-1H-pyrazol-3-yl)methyl)thiazole-4-carboxamide was obtained as a pale tan solid (18 mg, 12%).
IR uₘₐₓ (cm⁻¹) 3305, 3210, 3135, 3089, 1642, 1554, 1299, 1120, 966, 769, 652, 519;
¹H (400 MHz, DMSO-d₆) δ 12.83 (br s, 2H), 8.95 (br s, 1H), 7.74 (d, J=7.6 Hz, 2H), 7.57 (s, 1H), 7.41 (app t, J=7.6 Hz, 2H), 7.30 (t, J=7.4 Hz, 1H), 6.59 (s, 1H), 4.45 (d, J=5.6 Hz, 2H), 2.99 (s, 3H);
¹³C (101 MHz, DMSO-d₆) δ 166.23 (br), 157.80, 128.81, 127.69, 125.01, 112.56 (br), 100.97, 40.77, 35.66 (br) (4 missing);
HRMS: m/z (ESI) calcd for C₁₅H₁₆N₅O₃S₂, 378.0695 (M+H)⁺; found, 378.0703

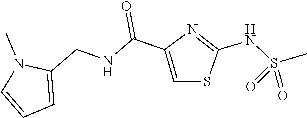 [SMILES: Cn1cccc1CNC(=O)c1csc(NS(C)(=O)=O)n1]

Following General Procedure B, using 2-(methylsulfonamido)thiazole-4-carboxylic acid (100 mg, 0.450 mmol) and 1-(1-methyl-1H-pyrrol-2-yl)methanamine (54 mg, 0.495 mmol) in anhydrous DMF (7 mL) at 20° C. for 20 h. The solvent was removed under high vacuum and the residue was dissolved in 5% aq. NaHCO₃ solution (30 mL) and extracted with EtOAc (2×30 mL). The aqueous phase was neutralised to pH 6-7 by dropwise addition of 1 M aq. HCl and a fine precipitate was removed by vacuum filtration. The filtrate was extracted with EtOAc (5×40 mL), and the combined organic extracts were washed with brine (35 mL), dried (MgSO₄), filtered and evaporated to give 2-(methylsulfonamido)-N-((1-methyl-1H-pyrrol-2-yl)methyl)thiazole-4-carboxamide as a pale tan solid (75 mg, 53%) after drying in a vacuum oven at 40° C. for 21 h.
IR uₘₐₓ (cm⁻¹): 3094, 2918, 1645, 1534, 1271, 1118, 967, 839, 557, 521;
¹H (400 MHz, DMSO-d₆) δ 12.68 (br s, 1H), 8.69 (br s, 1H), 7.54 (s, 1H), 6.68 (s, 1H), 5.97 (s, 1H), 5.87-5.93 (m, 1H), 4.38 (d, J=4.4 Hz, 2H), 3.55 (s, 3H), 2.95 (br s, 3H);
¹³C (101 MHz, DMSO-d₆) δ 166.46, 157.32, 128.49, 122.50, 112.32, 108.38, 106.20, 40.71, 34.54, 33.35; (1 missing);
HRMS: m/z (ESI) calcd for C₁₁H₁₄N₄NaO₃S₂, 337.0405 (M+Na)⁺; found, 337.0422

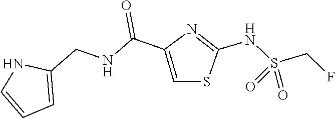 [SMILES: O=C(NCc1ccc[nH]1)c1csc(NS(=O)(=O)CF)n1]

Following General Procedure B, using 2-(fluoromethylsulfonamido)thiazole-4-carboxylic acid (76 mg, 0.316 mmol) and 1-(1H-pyrrol-2-yl)methanamine (33 mg, 0.348 mmol) in anhydrous MeCN (14 mL) at 20° C. for 20 h. Water (35 mL) was added, and the solution was saturated with NaCl then extracted with EtOAc (3×50 mL). The combined organic extracts were washed with brine (20 mL), dried (MgSO₄), filtered and evaporated to give a solid that was dissolved in sat. aq. NaHCO₃ solution (25 mL) and extracted with DCM (8×40 mL). The aqueous phase was acidified by dropwise addition of 32% aq. HCl and extracted with EtOAc (4×25 mL). The combined organic extracts were washed with brine (15 mL), dried (MgSO₄), filtered and evaporated to give 2-(fluoromethylsulfonamido)-N-((1H-pyrrol-2-yl)methyl)thiazole-4-carboxamide as a tan solid (102 mg, “102” %) after drying in an oven at 60° C. for 14 h. Traces of EtOAc were not diminished by further drying.
IR uₘₐₓ (cm⁻¹) 3328, 3097, 1644, 1531, 1271, 1127, 1116, 835, 728, 557;
¹H (400 MHz, DMSO-d₆) δ 13.30 (br s, 1H), 10.67 (s, 1H), 8.88 (s, 1H), 7.60 (s, 1H), 6.66 (s, 1H), 5.94 (s, 2H), 5.30 (d, J=46 Hz, 2H), 4.36 (d, J=5.4 Hz, 2H);
¹³C (101 MHz, DMSO-d₆) δ 169.46, 156.97, 132.41, 127.79, 117.30, 112.50, 107.36, 106.25, 90.72 (d, J=208 Hz), 36.04;
HRMS: m/z (ESI) calcd for C₁₀H₁₁FN₄NaO₃S₂, 341.0154 (M+Na)⁺; found, 341.0174

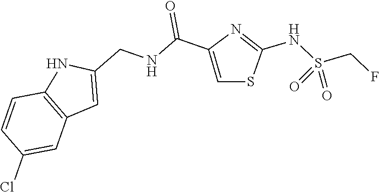 [SMILES: O=C(NCc1cc2cc(Cl)ccc2[nH]1)c1csc(NS(=O)(=O)CF)n1]

Following General Procedure B, using 2-(fluoromethylsulfonamido)thiazole-4-carboxylic acid (78 mg, 0.323 mmol) and 1-(5 (6)-chloro-1H-benzo[d]imidazol-2-yl)methanamine dihydrochloride (90 mg, 0.355 mmol) in anhydrous DMF (1.2 mL) and anhydrous MeCN (12 mL) at 20° C. for 96 h. Water (35 mL) was added and the solution was saturated with NaCl then extracted with EtOAc (3×50 mL). The combined organic extracts were washed with water (40 mL), brine (30 mL), dried (MgSO₄), filtered and evaporated to give a pale yellow solid. The aqueous washes were combined and extracted with EtOAc (5×80 mL). The combined organic extracts were washed with brine (40 mL), dried (MgSO₄), filtered and evaporated to give a yellow solid. The solids were combined and dissolved in sat. aq. NaHCO₃ solution (35 mL) and extracted with DCM (6×40 mL). The aqueous phase was neutralised by dropwise addition of 32% aq. HCl and extracted with EtOAc (4×50 mL). The combined organic extracts were washed with brine (35 mL), dried (MgSO₄), filtered and evaporated to give N-((5 (6)-chloro-1H-benzo[d]imidazol-2-yl)methyl)-2-(fluoromethylsulfonamido)thiazole-4-carboxamide as a pale tan solid (137 mg, “105” %) after drying in an oven at 60° C. for 20 h. Traces of DCM and EtOAc were not diminished by further drying.
IR uₘₐₓ (cm⁻¹) 3410, 3104, 2972, 1648, 1542, 1466, 1265, 1224, 1123, 840, 556;
¹H (400 MHz, DMSO-d₆) δ 9.05 (br s, 1H), 7.56 (s, 1H), 7.46-7.54 (m, 2H), 7.50 (s, 1H), 7.17 (d, J=8.5 Hz, 1H), 5.22 (d, J=47 Hz, 2H), 4.64 (d, J=5.1 Hz, 2H);
¹³C (101 MHz, DMSO-d₆) δ 169.41, 159.25, 153.60, 139.82, 136.93, 125.93, 121.83, 115.79, 114.73, 113.17, 90.40 (d, J=208 Hz), 37.28 (1 missing);
HRMS: m/z (ESI) calcd for C₁₃H₁₂ClFN₅O₃S₂, 404.0054 (M+H)⁺; found, 404.0068

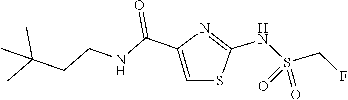 [SMILES: CC(C)(C)CCNC(=O)c1csc(NS(=O)(=O)CF)n1]

Following General Procedure B, using 2-(fluoromethylsulfonamido)thiazole-4-carboxylic acid (76 mg, 0.316 mmol) and 3,3-dimethylbutylamine (35 mg, 0.348 mmol) in anhydrous MeCN (12 mL) at 20° C. for 21 h. Water (30 mL) was added and the solution was cooled in an ice/water bath while NaCl was added. A solid was collected by vacuum filtration, washed with water (4×5 mL), and dried to give 38 mg of a white solid. Further solid was recovered by extraction of the filtrate with EtOAc (4×35 mL). The combined organic extracts were washed with brine (40 mL), dried (MgSO₄), filtered and evaporated to give a white solid that was dissolved in sat. aq. NaHCO₃ solution (35 mL) and extracted with DCM (6×40 mL). The aqueous phase was acidified by dropwise addition of 32% aq. HCl and extracted with EtOAc (4×40 mL). The combined organic extracts were washed with brine (30 mL), dried (MgSO₄), filtered and evaporated to give N-(3,3-dimethylbutyl)-2-(fluoromethylsulfonamido)thiazole-4-carboxamide (93 mg, 91%) as a white solid after drying in an oven at 60° C. for 16.5 h.
IR uₘₐₓ (cm⁻¹) 3328, 2955, 1646, 1524, 1283, 1126, 1059, 898, 659, 559, 518;
¹H (400 MHz, DMSO-d₆) δ 13.23 (br s, 1H), 8.53 (s, 1H), 7.49 (s, 1H), 5.30 (d, J=46 Hz, 2H), 3.21 (m, 2H), 1.41 (m, 2H), 0.91 (s, 9H);
¹³C (101 MHz, DMSO-d₆) δ 169.48, 157.06, 132.97, 111.82, 90.68 (d, J=207 Hz), 42.46, 35.60, 29.63, 29.19;
HRMS: m/z (ESI) calcd for C₁₁H₁₈FN₃NaO₃S₂, 346.0689 (M+Na)⁺; found, 346.0671

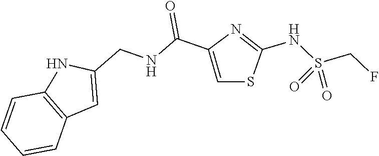 [SMILES: O=C(NCc1cc2ccccc2[nH]1)c1csc(NS(=O)(=O)CF)n1]

Following General Procedure B, using 2-(fluoromethylsulfonamido)thiazole-4-carboxylic acid (60 mg, 0.250 mmol) and 1-(1H-indol-2-yl)methanamine hydrochloride (50 mg, 0.275 mmol) in anhydrous MeCN (11 mL) and DMF (0.5 mL) at 20° C. for 19 h. Water (35 mL) was added and the solution was saturated with NaCl then extracted with EtOAc (3×40 mL). The combined organic extracts were washed with brine (30 mL), dried (MgSO₄), filtered and evaporated to give a pale yellow oil that was dissolved in sat. aq. NaHCO₃ solution (35 mL) and extracted with DCM (5×40 mL). The aqueous phase was neutralised by dropwise addition of 32% aq. HCl and extracted with EtOAc (4×40 mL). The combined organic extracts were washed with brine (35 mL), dried (MgSO₄), filtered and evaporated to give a solid that was again dissolved in sat. aq. NaHCO₃ solution (20 mL) and extracted with DCM (5×40 mL). The aqueous phase was neutralised by dropwise addition of 32% aq. HCl and extracted with EtOAc (5×40 mL). The combined organic extracts were washed with brine (35 mL), dried (MgSO₄), filtered and evaporated to give 2-(fluoromethylsulfonamido)-N-(1H-indol-2-ylmethyl)thiazole-4-carboxamide (86 mg, 93%) as a pale orange solid after drying in an oven at 60° C. for 14 h.
IR uₘₐₓ (cm⁻¹) 3387, 1634, 1538, 1471, 1457, 1424, 1271, 1233, 1128, 1057, 962, 840, 741, 559;
¹H (400 MHz, DMSO-d₆) δ 13.36 (br s, 1H), 10.93 (s, 1H), 8.57 (br s, 1H), 7.43 (d, J=7.8 Hz, 1H), 7.35 (br s, 1H), 7.33 (d, J=7.8 Hz, 1H), 7.02 (app t, J=7.8 Hz, 1H), 6.93 (app t, J=7.6 Hz, 1H), 6.26 (s, 1H), 5.11 (d, J=46 Hz, 2H), 4.54 (d, J=5.8 Hz, 2H);
¹³C (101 MHz, DMSO-d₆) δ 169.48, 160.05, 140.20 (br), 136.95, 136.12, 127.87, 120.67, 119.57, 118.84, 112.80, 111.11, 99.12, 90.16 (d, J=207 Hz), 36.21;
HRMS: m/z (ESI) calcd for C₁₄H₁₃FN₄NaO₃S₂, 391.0311 (M+Na)⁺; found, 391.0328

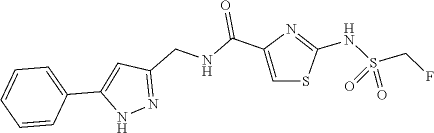 [SMILES: O=C(NCc1cc(-c2ccccc2)[nH]n1)c1csc(NS(=O)(=O)CF)n1]

Following General Procedure B, using 2-(fluoromethylsulfonamido)thiazole-4-carboxylic acid (38 mg, 0.158 mmol) and (5-phenyl-1H-pyrazol-3-yl)methanamine (32 mg, 0.185 mmol) in anhydrous MeCN (5 mL) at 50° C. for 26.5 h. After cooling to ambient temperature, water (20 mL) was added and the resultant suspension was cooled in an ice bath for 15 min. The solid was collected by vacuum-filtration, washed with water (3×5 mL), and dried in a vacuum oven at 40° C. for 16 h; 2-(fluoromethylsulfonamido)-N-((5-phenyl-1H-pyrazol-3-yl)methyl)thiazole-4-carboxamide (48 mg, 77%) was obtained as a pale tan solid.
IR uₘₐₓ (cm⁻¹) 3306, 3243, 3094, 2955, 1643, 1547, 1442, 1423, 1318, 1275, 1133, 1007, 893, 766, 666, 590, 508;
¹H (600 MHz, DMSO-d₆) δ13.16 (br s, 2H), 9.10 (br t, J=5.7 Hz, 1H), 7.74 (d, J=7.7 Hz, 2H), 7.62 (s, 1H), 7.41 (app t, J=7.5 Hz, 2H), 7.30 (t, J=7.4 Hz, 1H), 6.60 (s, 1H), 5.31 (d, J=46 Hz, 2H), 4.45 (d, J=5.7 Hz, 2H);
¹³C (151 MHz, DMSO-d₆) δ 169.46, 157.22, 144.00 (br), 132.38, 130.86 (br), 128.83, 127.72, 125.03, 112.63, 101.02, 90.73 (d, J=207 Hz), 35.76 (br) (1 missing);
HRMS: m/z (ESI) calcd for C₁₅H₁₅N₅O₃S₂F, 396.0600 (M+H)⁺; found, 396.0596

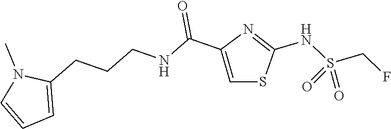 [SMILES: Cn1cccc1CCCNC(=O)c1csc(NS(=O)(=O)CF)n1]

Following General Procedure B, using 2-(fluoromethylsulfonamido)thiazole-4-carboxylic acid (75 mg, 0.312 mmol) and 1-(1-methyl-1H-pyrrol-2-yl)methanamine (38 mg, 0.343 mmol) in anhydrous MeCN (12 mL) at 20° C. for 23 h. Water (35 mL) was added, and the solution was saturated with NaCl then extracted with EtOAc (4×30 mL). The combined organic extracts were washed with brine (20 mL), dried (MgSO₄), filtered and evaporated to give an oil that was dissolved in sat. aq. NaHCO₃ solution (25 mL) and extracted with DCM (5×20 mL). The aqueous phase was acidified by dropwise addition of 32% aq. HCl and extracted with EtOAc (3×25 mL). The combined organic extracts were washed with brine (15 mL), dried (MgSO₄), filtered and evaporated to give 2-(fluoromethylsulfonamido)-N-((1-methyl-1H-pyrrol-2-yl)methyl)thiazole-4-carboxamide as a gummy tan solid (86 mg, 93%) after drying in a vacuum oven at 40° C. for 16 h.
IR uₘₐₓ (cm⁻¹) 3640, 3096, 1652, 1532, 1293, 1131, 834, 739, 657, 556;
¹H (400 MHz, DMSO-d₆) δ 13.30 (br s, 1H), 8.84 (s, 1H), 7.61 (s, 1H), 6.68 (s, 1H), 5.98 (s, 1H), 5.90 (s, 1H), 5.29 (d, J=46 Hz, 2H), 4.39 (br s, 2H), 3.55 (s, 3H);
¹³C (101 MHz, DMSO-d₆) δ 169.51, 156.75, 132.17, 128.30, 122.56, 112.59, 108.49, 106.21, 90.70 (d, J=208 Hz), 34.63, 33.35;
HRMS: m/z (ESI) calcd for C₁₁H₁₃FN₄NaO₃S₂, 355.0311 (M+Na)⁺; found, 355.0324

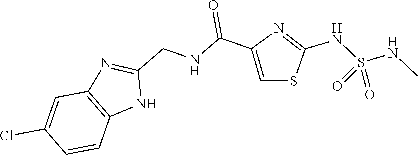 [SMILES: CNS(=O)(=O)Nc1nc(C(=O)NCc2nc3cc(Cl)ccc3[nH]2)cs1]

Following General Procedure B, using 2-((N-methylsulfamoyl)amino)thiazole-4-carboxylic acid (94 mg, 0.396 mmol) and 1-(5 (6)-chloro-1H-benzo[d]imidazol-2-yl)methanamine dihydrochloride (122 mg, 0.479 mmol) in anhydrous MeCN (10 mL) at 50° C. for 66.5 h. After cooling to ambient temperature, water (80 mL) was added and the resultant suspension was cooled in an ice bath for 30 min. The liquid was decanted and the solid residue was dissolved in MeOH (15 mL). The MeOH was evaporated and the orange residue was dried in a vacuum oven at 40° C. for 13 h. The dried crude material was dissolved in sat. aq. NaHCO₃ (10 mL), and the aqueous solution was extracted with EtOAc (2×10 mL). The aqueous phase was acidified to pH=6 with 2 M aq. HCl, then extracted with DCM (10 mL) and THF (3×10 mL). The combined THF extracts were dried (MgSO₄), filtered, and evaporated to leave a viscous, orange oil, which was dried in a vacuum oven at 40° C. for 14 h. N-((5 (6)-chloro-1H-benzo[d]imidazol-2-yl)methyl)-2-((N-methylsulfamoyl)amino)thiazole-4-carboxamide (59 mg, 37%) was obtained as a tan solid.
IR uₘₐₓ (cm⁻¹) 3245, 3098, 2968, 1649, 1543, 1454, 1260, 1116, 1059, 883, 805, 562, 461;
¹H (400 MHz, CD₃OD) δ 7.67 (s, 1H), 7.50 (d, J=2.0 Hz, 1H), 7.46 (d, J=8.6 Hz, 1H), 7.18 (dd, J=8.6, 1.9 Hz, 1H), 4.80 (s, 2H), 2.65 (s, 3H);
¹³C (101 MHz, CD₃OD) δ 163.24, 163.13, 154.72, 144.47, 140.32, 137.97, 129.14, 124.00, 117.83, 116.68, 115.66, 38.33, 29.35;
HRMS: m/z (ESI) calcd for C₁₃H₁₄N₆O₃S₂³⁵Cl, 410.0257 (M+H)⁺; found, 401.0257

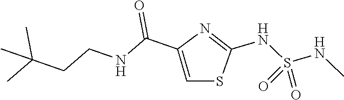 [SMILES: CNS(=O)(=O)Nc1nc(C(=O)NCCC(C)(C)C)cs1]

Following General Procedure B, using 2-((N-methylsulfamoyl)amino)thiazole-4-carboxylic acid (95 mg, 0.400 mmol) and 3,3-dimethylbutylamine (49 mg, 65 μL, 0.483 mmol) in anhydrous MeCN (10 mL) at 50° C. for 18 h. Water (20 mL) and brine (10 mL) were added, and the resultant mixture was cooled to ambient temperature. The mixture was extracted with EtOAc (2×15 mL), and the combined organic extracts were evaporated to leave a pink oil, which was dissolved in sat. aq. NaHCO₃ (10 mL). The aqueous solution was extracted with DCM (3×15 mL) and acidified to pH ≈1 with 2 M aq. HCl. The aqueous acid was saturated with NaCl and extracted with THF (2×15 mL). The combined THF extracts were dried (MgSO₄), filtered, and evaporated to leave N-(3,3-dimethylbutyl)-2-((N-methylsulfamoyl)amino)thiazole-4-carboxamide as an off-white solid (120 mg, 94%).
IR uₘₐₓ (cm⁻¹) 3345, 3253, 2957, 1646, 1565, 1530, 1278, 1248, 1175, 1129, 1041, 840, 667, 556, 492;
¹H (400 MHz, CD₃OD) δ 7.61 (s, 1H), 3.40-3.34 (m, 2H), 2.65 (s, 3H), 1.54-1.48 (m, 2H), 0.96 (s, 9H);
¹³C (101 MHz, CD₃OD) δ 163.07, 162.29, 144.09, 116.76, 43.89, 37.10, 30.60, 29.68, 29.28;
HRMS: m/z (ESI) calcd for C₁₁H₂₀N₄NaO₃S₂, 343.0875 (M+Na)⁺; found, 343.0876

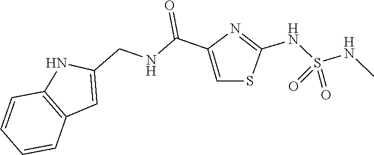 [SMILES: CNS(=O)(=O)Nc1nc(C(=O)NCc2cc3ccccc3[nH]2)cs1]

Following General Procedure B, using 2-((N-methylsulfamoyl)amino)thiazole-4-carboxylic acid (125 mg, 0.527 mmol) and (1H-indol-2-yl)methanamine hydrochloride (115 mg, 0.630 mmol) in anhydrous MeCN (10 mL) at 50° C. for 23 h. After cooling to ambient temperature, water (80 mL) was added and the resultant cloudy mixture was cooled in an ice bath for 30 min. The liquid was decanted, and the solid residue was dissolved in MeOH (25 mL). The MeOH was evaporated, and the orange residue was dried in a vacuum oven at 40° C. for 16 h. The dried solid was partitioned between DCM (15 mL) and sat. aq. NaHCO₃ (15 mL), the phases were separated, and the aqueous phase was washed with DCM (15 mL). The aqueous portion was acidified to pH ≈1 with 2 M aq. HCl, and the resultant precipitate was collected by vacuum-filtration. The solid was washed with water (3×15 mL) then dried in a vacuum oven at 40° C. for 15 h; N-((1H-indol-2-yl)methyl)-2-((N-methylsulfamoyl)amino)thiazole-4-carboxamide was obtained as an off-white solid (46 mg, 24%).
IR uₘₐₓ (cm⁻¹) 3300, 1652, 1539, 1340, 1289, 1122, 884, 794, 750, 660, 568, 466;
¹H (400 MHz, CD₃OD) δ 7.68 (s, 1H), 7.45 (d, J=7.9 Hz, 1H), 7.31 (d, J=8.1 Hz, 1H), 7.05 (app t, J=7.6 Hz, 1H), 6.96 (app t, J=7.5 Hz, 1H), 6.35 (s, 1H), 4.69 (s, 2H), 2.63 (s, 3H);
¹³C (101 MHz, CD₃OD) δ 162.84 (br), 138.16, 136.83, 136.74*, 129.68, 129.60*, 122.28, 120.90, 120.87*, 120.18, 117.43 (br), 111.90, 101.07, 37.84, 37.82*, 29.28 (2 missing);
Peaks appear at approx. half height of their close neighbours; all are (indol-2-yl)methyl-associated and likely belong to rotamers
HRMS: m/z (ESI) calcd for C₁₄H₁₅N₅NaO₃S₂, 388.0514 (M+Na)⁺; found, 388.0505

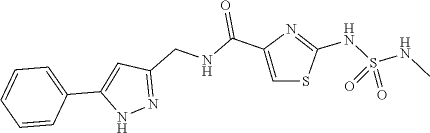 [SMILES: CNS(=O)(=O)Nc1nc(C(=O)NCc2cc(-c3ccccc3)[nH]n2)cs1]

Following General Procedure B, using 2-((N-methylsulfamoyl)amino)thiazole-4-carboxylic acid (110 mg, 0.464 mmol) and (5-phenyl-1H-pyrazol-3-yl)methanamine (94 mg, 0.543 mmol) in anhydrous MeCN (10 mL) at 50° C. for 40 h. After cooling to ambient temperature, water (60 mL) was added and the resultant suspension was cooled in an ice bath for 30 min. The solid was collected by vacuum-filtration, washed with water (3×10 mL), and dried in a vacuum oven at 40° C. for 69 h. The dried solid was suspended in sat. aq. NaHCO₃ (20 mL) and stirred prior to washing with EtOAc (2×20 mL). The aqueous phase was acidified to pH ≈1 with 2 M aq. HCl, saturated with NaCl, and extracted with THF (2×15 mL). The combined THF extracts were washed with brine (10 mL), dried (MgSO₄), filtered, and evaporated to leave 2-((N-methylsulfamoyl)amino)-N-((5-phenyl-1H-pyrazol-3-yl)methyl)thiazole-4-carboxamide as an off-white solid (91 mg, 50%).
IR uₘₐₓ (cm⁻¹) 3287, 3183, 3134, 1742, 1648, 1539, 1306, 1122, 883, 769, 695, 647, 574;
¹H (400 MHz, CD₃OD) δ 7.71-7.69 (m, 3H), 7.41 (app t, J=7.6 Hz, 2H), 7.32 (t, J=7.4 Hz, 1H), 6.60 (s, 1H), 4.60 (s, 2H), 2.64 (s, 3H);
¹³C (101 MHz, DMSO-d₆) δ 159.17 (br), 146.86 (br), 146.18 (br), 131.21 (br), 128.83, 127.74, 125.06, 114.74 (br), 100.97, 35.58, 28.78 (2 missing);
HRMS: m/z (ESI) calcd for C₁₅H₁₇N₆O₃S₂, 393.0804 ([M+H]⁺); found, 393.0807

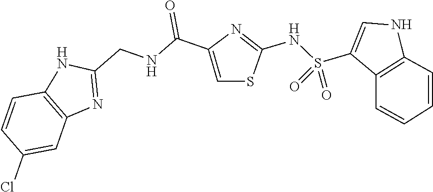 [SMILES: O=C(NCc1nc2cc(Cl)ccc2[nH]1)c1csc(NS(=O)(=O)c2c[nH]c3ccccc23)n1]

Following General Procedure B, using 2-(1H-indole-3-sulfonamido)thiazole-4-carboxylic acid (100 mg, 0.309 mmol) and (5-chloro-1H-1,3-benzodiazol-2-yl)methanamine dihydrochloride (94 mg, 0.369 mmol) in anhydrous MeCN (10 mL) at 50° C. for 20 h. After cooling to ambient temperature, water (40 mL) was added and the resultant suspension was cooled in an ice bath for 30 min. The solid was collected by vacuum-filtration, washed with water (3×15 mL), and dried in a vacuum oven at 40° C. for 13.5 h. The crude material was dissolved in a mixture of THF (5 mL) and EtOAc (10 mL), and washed with water (2×10 mL). The organic portion was washed with sat. aq. NaHCO₃ (3×5 mL), then diluted with diethyl ether (20 mL). A dark yellow oil came out of solution, and the mixture was extracted with water (10 mL). This water wash was acidified to pH ≈5 with 1 M aq. HCl, and the resultant precipitate was collected by vacuum-filtration. The collected solid was washed with water (3×5 mL), and dried in a vacuum oven at 40° C. for 17 h; N-((5-chloro-1H-benzo[d]imidazol-2-yl)methyl)-2-(1H-indole-3-sulfonamido)thiazole-4-carboxamide (39 mg, 26%) was obtained as an off-white solid.
IR uₘₐₓ (cm⁻¹) 3275, 3115, 1658, 1541, 1420, 1243, 1114, 1019, 882, 746, 676, 588, 425;
¹H (400 MHz, CD₃OD) δ 7.95 (s, 1H), 7.87 (d, J=7.5 Hz, 1H), 7.57 (s, 1H), 7.47 (d, J=1.9 Hz, 1H), 7.46-7.42 (m, 2H), 7.25-7.21 (m, 1H), 7.20-7.16 (m, 2H), 4.73 (s, 2H);
¹³C (101 MHz, CD₃OD) δ 163.83, 162.48, 154.55, 141.91, 140.00, 137.98, 137.68, 132.06, 129.31, 124.64, 124.50, 124.16, 122.77, 120.52, 117.34, 116.64, 115.61, 114.43, 113.39, 38.25;
HRMS: m/z (ESI) calcd for C₂₀H₁₆³⁵ClN₆O₃S₂, 487.0414 ([M+H]⁺); found, 487.0413

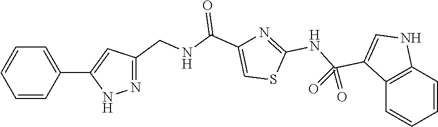

Following General Procedure B, using 2-(1H-indole-3-sulfonamido)thiazole-4-carboxylic acid (84 mg, 0.260 mmol) and (5-phenyl-1H-pyrazol-3-yl)methanamine (55 mg, 0.318 mmol) in anhydrous MeCN (9 mL) at 50° C. for 22 h. After cooling to ambient temperature, water (15 mL) was added and the resultant suspension transferred to a beaker. Residual solid in the reaction flask was rinsed into the beaker with water (15 mL). The suspension was stirred and cooled in an ice bath for 30 min. The solid was collected by vacuum-filtration, washed with water (3×15 mL), and dried in a vacuum oven at 40° C. for 20 h; 2-(1H-indole-3-sulfonamido)-N-((5-phenyl-1H-pyrazol-3-yl)methyl)thiazole-4-carboxamide (105 mg, 84%) was obtained as an off-white solid.
IR uₘₐₓ (cm⁻¹) 3297, 3105, 1642, 1546, 1509, 1424, 1289, 1113, 1014, 880, 745, 679, 653, 589;
¹H (400 MHz, DMSO-d₆) δ 12.80 (br s, 2H), 11.90 (s, 1H), 8.76 (br s, 1H), 7.96 (s, 1H), 7.76 (d, J=8.0 Hz, 1H), 7.72 (d, J=7.7 Hz, 2H), 7.54 (s, 1H), 7.47 (d, J=8.0 Hz, 1H), 7.39 (app t, J=7.6 Hz, 2H), 7.29 (t, J=7.4 Hz, 1H), 7.21 (app t, J=7.6 Hz, 1H), 7.16 (app t, J=7.5 Hz, 1H), 6.55 (s, 1H), 4.40 (d, J=5.6 Hz, 2H);
¹³C (101 MHz, DMSO-d₆) δ 136.16, 129.90 (br) 128.80, 127.68, 125.01, 123.22, 122.78, 121.02, 119.43, 112.50, 100.91, 35.67 (br) (8 missing);
HRMS: m/z (ESI) calcd for C₂₂H₁₉N₆O₃S₂, 479.0960 ([M+H]⁺); found, 479.0962

### General Procedure C.

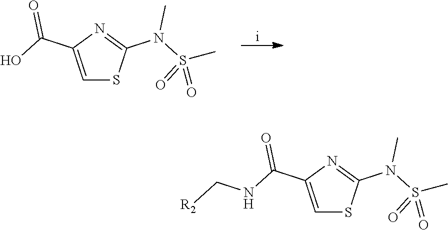

### i; Amine, DIPEA, HATU, MeCN, 20° C. or 50° C.

To a stirred suspension of 2-(N,S-dimethylsulfonamido)thiazole-4-carboxylic acid (1.0 eq.) in anhydrous MeCN was added DIPEA (2.0 eq.) under an inert atmosphere. Once the solid was fully dissolved, HATU (1.0 eq.) was added, followed by an amine (1.05-1.2 eq.). The reaction mixture was stirred at 20° C. or 50° C. Work-up procedures varied and are detailed for each compound.

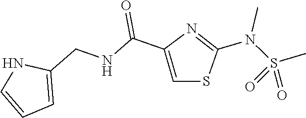 [SMILES: CN(c1nc(C(=O)NCc2ccc[nH]2)cs1)S(C)(=O)=O]

Following General Procedure C, using 2-(N,S-dimethylsulfonamido)thiazole-4-carboxylic acid (81 mg, 0.344 mmol) and 1-(1H-pyrrol-2-yl)methanamine (36 mg, 0.378 mmol) in anhydrous MeCN (6.5 mL) at 20° C. for 70 h. The reaction mixture was diluted with water (45 mL) and cooled to 0-5° C. for 1 h. The precipitate was collected by vacuum filtration, washed with water (3×10 mL) and dried in an oven at 60° C. for 5 h to give N-((1H-pyrrol-2-yl)methyl)-2-(N,S-dimethylsulfonamido)thiazole-4-carboxamide as an off-white solid (92 mg, 85%).
IR uₘₐₓ (cm⁻¹) 3412, 3303, 1665, 1552, 1488, 1349, 1150, 977, 744, 514;
¹H (400 MHz, DMSO-d₆) δ 10.55 (s, 1H), 8.43 (t, J=5.4 Hz, 1H), 7.88 (s, 1H), 6.63 (s, 1H), 5.90 (s, 2H), 4.39 (d, J=5.9 Hz, 2H), 3.49 (s, 3H), 3.27 (s, 3H);
¹³C (101 MHz, DMSO-d₆) δ 160.91, 160.00, 145.08, 128.82, 119.85, 117.19, 107.08, 105.92, 37.07, 36.24, 35.57;
HRMS: m/z (ESI) calcd for C₁₁H₁₄N₄NaO₃S₂, 337.0405 (M+Na)⁺; found, 337.0421

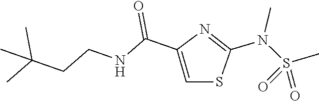 [SMILES: CN(c1nc(C(=O)NCCC(C)(C)C)cs1)S(C)(=O)=O]

Following General Procedure C, using 2-(N,S-dimethylsulfonamido)thiazole-4-carboxylic acid (84 mg, 0.356 mmol) and 3,3-dimethylbutylamine (38 mg, 50 μL, 0.372 mmol) in anhydrous MeCN (5 mL) at 50° C. for 15.5 h. Water (40 mL) was added and the resultant mixture was cooled to ambient temperature. The aqueous solution was decanted, and the residual oil was dissolved in EtOAc (15 mL). The organic solution was dried (MgSO₄), filtered, and evaporated to leave a viscous, orange-yellow oil. The decanted aqueous solution was extracted with EtOAc (2×25 mL), and the combined EtOAc extracts were washed with 1 M aq. HCl (20 mL), sat. aq. NaHCO₃ (20 mL), water (20 mL), and brine (20 mL). The organic phase was dried (MgSO₄), filtered, and evaporated to leave a pale yellow, viscous oil. The two isolated oils were combined and purified by flash chromatography (SiO₂, petroleum ether 40/60:EtOAc, 1:1). The oil thus isolated was dried in a vacuum oven at 40° C. for 13.5 h; N-(3,3-dimethylbutyl)-2-(N,S-dimethylsulfonamido)thiazole-4-carboxamide was obtained as a white solid (106 mg, 93%).
IR uₘₐₓ (cm⁻¹) 3415, 3397, 3119, 2996, 2951, 2916, 2866, 1657, 1557, 1491, 1347, 1156, 765, 514, 487;
¹H (600 MHz, CDCl₃) δ 7.69 (s, 1H), 7.03 (br t, J=5.9 Hz, 1H), 3.49 (s, 3H), 3.41-3.37 (m, 2H), 3.01 (s, 3H), 1.50-1.47 (m, 2H), 0.92 (s, 9H);
¹³C (151 MHz, CDCl₃) δ 161.08, 160.55, 145.84, 119.12, 43.35, 37.45, 36.51, 36.03, 30.00, 29.43;
HRMS: m/z (ESI) calcd for C₁₂H₂₁N₃NaO₃S₂, 342.0922 (M+Na)⁺; found, 342.0925

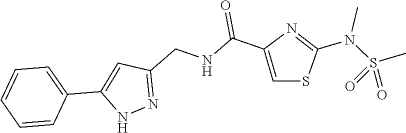 [SMILES: CN(c1nc(C(=O)NCc2cc(-c3ccccc3)[nH]n2)cs1)S(C)(=O)=O]

Following General Procedure C, using 2-(N,S-dimethylsulfonamido)thiazole-4-carboxylic acid (88 mg, 0.372 mmol) and (5-phenyl-1H-pyrazol-3-yl)methanamine (77 mg, 0.445 mmol) in anhydrous MeCN (5 mL) at 50° C. for 19.5 h. Water (40 mL) was added and the resultant mixture was cooled to ambient temperature, then in an ice bath for 1 h. The aqueous solution was decanted, and the residual oil was dissolved in EtOAc (15 mL). The organic solution was dried (MgSO₄), filtered, and evaporated to leave a viscous, yellow-orange oil, which was purified by flash chromatography (SiO₂, DCM:MeOH, 92:8); 2-(N,S-dimethylsulfonamido)-N-((5-phenyl-1H-pyrazol-3-yl)methyl)thiazole-4-carboxamide was obtained as a white solid (106 mg, 93%).
IR uₘₐₓ (cm⁻¹) 3184, 3139, 3115, 3017, 1652, 1553, 1486, 1352, 1156, 959, 776, 748, 699, 654, 512, 495;
¹H (400 MHz, CDCl₃) δ 7.89 (br t, J=5.5 Hz, 1H), 7.77 (s, 1H), 7.66 (d, J=7.6 Hz, 2H), 7.37 (app t, J=7.5 Hz, 2H), 7.30 (t, J=7.4 Hz, 1H), 6.50 (s, 1H), 4.68 (d, J=5.5 Hz, 2H), 3.41 (s, 3H), 2.96 (s, 3H);
¹³C (101 MHz, CDCl₃) δ 161.41, 161.25, 147.71 (br), 146.38 (br), 145.14, 131.19, 128.92, 128.25, 125.54, 120.01, 101.70, 37.47, 36.43, 35.97;
HRMS: m/z (ESI) calcd for C₁₆H₁₈N₅O₃S₂, 392.0851 ([M+H]⁺); found, 392.0849

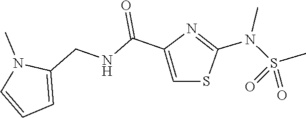 [SMILES: CN(c1nc(C(=O)NCc2cccn2C)cs1)S(C)(=O)=O]

Following General Procedure C, using 2-(N,S-dimethylsulfonamido)thiazole-4-carboxylic acid (81 mg, 0.344 mmol) and 1-(1-methyl-1H-pyrrol-2-yl)methanamine (42 mg, 0.378 mmol) in anhydrous MeCN (5.7 mL) at 20° C. for 20 h. The reaction mixture was diluted with water (40 mL), saturated with NaCl, and extracted with EtOAc (3×30 mL). The combined organic extracts were washed with brine (20 mL), dried (MgSO₄), filtered and evaporated to give a viscous oil that was purified by flash chromatography (SiO₂, DCM/MeOH, 98:2) to give N-((1-methyl-1H-pyrrol-2-yl)methyl)-2-(N,S-dimethylsulfonamido)thiazole-4-carboxamide as a gummy white solid (76 mg, 68%) after drying under high vacuum at 20° C. for 8.5 h.
IR uₘₐₓ (cm⁻¹) 3394, 3076, 2924, 1669, 1546, 1483, 1346, 1151, 965, 653, 513;
¹H (400 MHz, CDCl₃) δ 7.81 (s, 1H), 7.10-7.19 (m, 1H), 6.65 (s, 1H), 6.16 (s, 1H), 6.08-6.13 (m, 1H), 4.63 (d, J=5.6 Hz, 2H), 3.62 (s, 3H), 3.52 (s, 3H), 3.03 (s, 3H);
¹³C (101 MHz, CDCl₃) δ 161.09, 159.97, 145.19, 128.37, 123.14, 119.85, 108.86, 106.84, 37.26, 36.50, 34.84, 33.81;
HRMS: m/z (ESI) calcd for C₁₂H₁₆N₄NaO₃S₂, 351.0562 (M+Na)⁺; found, 351.0590

### Example 3—Biological Assays

dGAE Assembly and Measuring the Pharmacological Activity of Inhibitor Compounds by Immunodetection

### (a) Sandwich ELISA

Recombinant dGAE was produced in a bacterial expression system and assembled in the presence of 10 mM DTT by incubation at 37° C. with agitation for 24 h, as described previously [6]. For testing the pharmacological activity, assembly reactions were performed with 100 μM dGAE and 500 μM tau aggregation inhibitor (Compound 9 or HMT) in Protein Lobind tubes (Eppendorf). Compound 9 was dissolved in DMSO (final 0.5%) and HMT in 10 mM phosphate buffer (PB) supplemented with 10 mM DTT. Control tubes with dGAE+DTT in PB and dGAE+DTT in 0.5% DMSO were also set up to allow for comparison of the extent of aggregation. Following a 24 h assembly reaction, the contents from each tube were mixed by pipetting and samples removed for the immunodetection of exposed epitopes using a solution-phase sandwich ELISA.

Ninety-six well Nunc Maxisorp plates were pre-coated with mAb 423 (10 μg/ml), which specifically recognises tau that is C-terminally truncated at Glu391 in the Pronase-resistant PHF core [42], washed three times with PBS containing 0.1% Tween 20 (PBST) and then blocked with 2% dried milk (Marvel) in PBS. Doubling dilutions of dGAE assembly samples were added from starting concentrations of 10 μg/ml in PBS and incubated for 1 h at 37° C. Bound tau was then detected using scAbs (10 μg/ml) and incubated for 1 h at 37° C. Anti-human C kappa HRP-conjugated secondary antibody was diluted 1:1,000 in 2% dried milk in PBS and incubated for 1 h at 37° C. Pierce 1-Step Ultra TMB-ELISA Substrate Solution was used to develop the reaction which was subsequently stopped with the addition of 1 M H₂SO₄. Absorbance was measured at 450 nm.

### (b) Cellular Tau Aggregation Inhibition Assay

A cell-based tau aggregation assay was performed as described previously [13, 25]. The process is described in more detail in WO 02/055720. In essence, fibroblast cells (3T6) express full-length tau (“T40”; the htau40 isoform) under control of an inducible promotor, and low constitutive levels of the PHF-core tau fragment (12-kDa fragment). When T40 expression is induced, it undergoes aggregation-dependent truncation within the cell, N-terminally at approximately amino acid residue 295 and C-terminally at approximately residue 390, thereby producing higher levels of the 12-kDa PHF-core domain fragment. Production of the 12-kDa fragment can be blocked in a dose-dependent manner by tau-aggregation inhibitors. Indeed, the quantitation of inhibitory activity of compounds with respect to proteolytic generation of the 12-kDa fragment within cells can be described entirely in terms of the same parameters which describe inhibition of tau-tau binding in vitro. That is, the extent of proteolytic generation of the 12-kDa fragment within cells is determined entirely by the extent to tau-tau binding through the repeat domain. The availability of the relevant proteases within the cell is non-limiting.

Compounds were tested initially at a single concentration of 2 μM, at least in triplicate, on the formation within cells of truncated tau detected on immunoblots of cell lysates separated by SDS-PAGE [25]. MTC was included as a reference tau aggregation inhibitor. For single concentration tests. The ratio of the quantity of both of the protein bands at different concentrations of TAI is measured, to correct for the level of expression of full-length tau and for cell density, particularly at high concentration of TAIs.

The inhibitory ratio (IR) is calculated by the equation:

$${IR}^{x} = [ \frac{{S}_{trunc . tau}^{x}}{{S}_{full tau}^{x}} ] \div [ \frac{{S}_{trunc . tau}^{0}}{{S}_{full tau}^{0}} ]$$

Where IR^{x}=inhibitory ratio for compound at x mM; S=signal on immunoblot (mean) for truncated tau (trunc. tau) or full-length tau (full tau) bands for compound at 0 or x mM. A ratio of 1.0 indicates no effect, whereas values decreasing from 1.0 indicate increasing inhibitory action. A curve is generated for different concentrations and the EC₅₀ value for the TAI is defined as the concentration at which the ratio of truncated tau: full-length tau is 50% of that ratio measured in the absence of TAI; this is determined graphically.

Compound 9 and MTC were further tested over a range of concentrations (0-20 μM for compound 9 and 0-2 μM for MTC; FIG. 2) and the ratio of the lower, truncated 12-kDa tau to the full-length T40 band was calculated. The concentration at which there is 50% inhibition of the 12-kDa band relative to T40 (referred to as the EC₅₀ value) was calculated from a graph of the protein band ratios relative to those observed in untreated cells [25].

### (c) In Vitro Cell-Free Assay for Establishing B₅₀

This is described in detail in WO 96/30766 and in [25]. Briefly, a fragment of tau corresponding to the core repeat domain, which has been adsorbed to a solid-phase substrate, is able to capture soluble, full-length tau and bind tau with high affinity. This association confers stability against proteolytic digestion of the aggregated tau molecules. The process is self-propagating and can be blocked selectively by prototype pharmaceutical agents [12].

More specifically, truncated tau (residues 297-390; dGA) diluted in carbonate buffer (pH 9.6) was bound to the assay plate, and another truncated tau species (residues 297-391; dGAE) was added in the aqueous phase. The aqueous phase binding buffer contained 0.05% Tween-20 and 1% gelatine in phosphate-buffered saline (pH 7.4). Bound dGAE was detected using mAb 423 that recognises a Glu-391 dependent epitope that is absent from the solid-phase dGA.

The concentration of compound required to inhibit the tau-tau binding by 50% is referred to as the B₅₀ value.

### (d) Toxicity in Cells—LD₅₀

Toxicity of the compounds described herein was assessed using the fibroblast cells used in the cell-based assay described above to assess EC₅₀. Toxicity was measured by cell numbers after 24 hrs exposure to the compound using a lactate dehydrogenase assay kit TOX-7 (Sigma Biosciences) according to the manufacturer's instructions after lysis of remaining cells. Alternatively, a kit from Promega UK (CytoTox 96) was used, again according to the manufacturer's instructions. LD₅₀ is determined as the concentration of compound at which 50% of cells are killed.

### Biological Data

| | Cell-based tau-tau assay | | | | |
|---|---|---|---|---|---|
| | Single concentration test | | Full assay | | |
| | Concentration | Truncated tau/ | EC₅₀ (μM) | B₅₀ (μM) | LD₅₀ (μM) |
| Compound | tested (μM) | T40 ratio (n = 3) | (n) | (n) | (n) |
| 1 | 10 | 5.013 ± 0.404 | NT | | 247 ± 16 (3) |
| 2 | 10 | 0.864 ± 0.059 | NT | | 253 ± 40 (3) |
| 3 | 10 | 2.282 ± 0.449 | NT | | 212 ± 14 (2) |
| 4 | 10 | 4.802 ± 1.138 | NT | | 264 ± 27 (3) |
| 5 | 10 | 0.727 ± 0.113 | NE | | 252 ± 23 (4) |
| 6 | 10 | 0.888 ± 0.057 | NT | | 217 ± 2.7 |
| | | | | | (3) |
| 7 | 10 | 0.444 ± 0.208 | NT | | 216 ± 46 (3) |
| 8 | 10 | 0.078 ± 0.014 | NT | | 204 ± 39 (3) |
| 9 | 2 | 0.266 ± 0.061 | 4.816 ± 1.02 | NE (>500 | 80.1 ± 45 |
| | | | (n = 10) | μM) (1) | (3) |
| 10 | 10 | 0.496 ± 0.096 | 29 | | 191 ± 26 (4) |
| | | | (n = 1) | | |
| 11 | 10 | 0.917 ± 0.188 | NT | | 222 ± 35 (3) |
| 12 | 20 | 1.007 ± 0.088 | NT | | 197 ± 11.93 |
| | | | | | (3) |
| 13 | 20 | 1.121 ± 0.162 | NT | | 197 ± 8.1 |
| | | | | | (3) |
| 14 | 2 | 0.381 ± 0.146 | 3.7 (1) | | 263 ± 39, |
| | | | | | n = 3 |
| 15 | 10 | 1.196 ± 0.257 | NT | | 221 ± 35 (3) |
| 16 | 20 | 0.628 ± 0.051 | NT | | 236 ± 25 (3) |
| 17 | 2 | 0.532 ± 0.150 | NT | | 228 ± 40 (3) |
| 18 | 10 | 0.141 ± 0.039 | NE | | 225 ± 38 (3) |
| 19 | 10 | 1.968 ± 0.946 | NT | | 231 ± 38 (3) |
| 20 | 10 | 0.803 ± 0.424 | NT | | 186 ± 13 (3) |
| 21 | 10 | 2.168 ± 0.856 | NT | | 227 ± 30 (4) |
| 22 | 10 | 1.356 ± 0.591 | NT | | 228 ± 36 (3) |
| 23 | 10 | 0.985 ± 0.101 | NE | | 226 ± 40 (3) |
| 24 | 10 | 3.487 ± 1.060 | NE | | 235 ± 44 (3) |
| 25 | 20 | 0.911 ± 0.100 | NE | 612.3 (1) | 202 ± 9.3 |
| | | | | | (3) |
| 26 | 20 | 1.319 ± 0.084 | NE | 616.3 (1) | 188 ± 11 (3) |

Compounds were tested in cellular tau aggregation assay, initially at a single concentration of 2-25 μM, as shown, and the result expressed as the ratio between truncated tau/full-length tau compared with untreated cells. A value of approximately 1.0 being negative and a value of <0.5 being a positive inhibitor. For certain compounds, tau aggregation appeared to be enhanced (where the ratio increased to >2.0).
NE, no effect; NT, not tested.

### REFERENCES

A number of publications are cited above in order to more fully describe and disclose the invention and the state of the art to which the invention pertains. Full citations for these references are provided below. The entirety of each of these references is incorporated herein.
1. G. K. Wilcock, M. M. Esiri, Plaques, tangles and dementia. A quantitative study. J. Neurol. Sci. 56, 343-356 (1982).
2. P. V. Arriagada, J. H. Growdon, E. T. Hedley-Whyte, B. T. Hyman, Neurofibrillary tangles but not senile plaques parallel duration and severity of Alzheimer's disease. Neurology. 42, 631-639 (1992).
3. R. Smith, A. Puschmann, M. Scholl, T. Ohlsson, J. van Swieten, M. Honer, E. Englund, O. Hansson, 18F-AV-1451 tau PET imaging correlates strongly with tau neuropathology in MAPT mutation carriers. Brain. 139, 2372-2379 (2016).
4. B. O. Schelter, H. Shiells, T. C. Baddeley, C. M. Rubino, H. Ganesan, J. Hammel, V. Vuksanovic, R. T. Staff, A. D. Murray, L. Bracoud, G. Riedel, S. Gauthier, J. Jia, P. Bentham, K. Kook, J. M. D. Storey, C. R. Harrington, C. M. Wischik, Concentration-dependent activity of hydromethylthionine on cognitive decline and brain atrophy in mild to moderate Alzheimer's disease. J. Alzheimer's Dis. 72, 931-946 (2019).
5. H. Shiells, B. O. Schelter, P. Bentham, T. C. Baddeley, C. M. Rubino, H. Ganesan, J. Hammel, V. Vuksanovic, R. T. Staff, A. D. Murray, L. Bracoud, D. J. Wischik, G. Riedel, S. Gauthier, J. Jia, H. J. Moebius, J. Hardlund, C. M. Kipps, K. Kook, J. M. D. Storey, C. R. Harrington, C. M. Wischik, Concentration-dependent activity of hydromethylthionine on clinical decline and brain atrophy in a randomized controlled trial in behavioral variant frontotemporal dementia. J. Alzheimer's Dis. 75, 501-519 (2020).
6. Y. K. Al-Hilaly, S. J. Pollack, D. M. Vadukul, F. Citossi, J. E. Rickard, M. Simpson, J. M. D. Storey, C. R. Harrington, C. M. Wischik, L. C. Serpell, Alzheimer's disease-like paired helical filament assembly from truncated tau protein is independent of disulfide crosslinking. J. Mol. Biol. 429, 3650-3665 (2017).
7. S. Lovestam, F. A. Koh, B. van Knippenberg, A. Kotecha, A. G. Murzin, M. Goedert, S. H. W. Scheres, Assembly of recombinant tau into filaments identical to those of Alzheimer's disease and chronic traumatic encephalopathy. eLife 11, e76494 (2022)
8. Y. K. Al-Hilaly, B. E. Foster, L. Biasetti, L. Lutter, S. J. Pollack, J. E. Rickard, J. M. D. Storey, C. R. Harrington, W. Xue, C. M. Wischik, L. C. Serpell, Tau (297-391) forms filaments that structurally mimic the core of paired helical filaments in Alzheimer's disease brain. FEBS Lett. 594, 944-950 (2020).
9. L. Lutter, Y. K. Al-Hilaly, C. J. Serpell, M. F. Tuite, C. M. Wischik, L. C. Serpell, W.-F. Xue, Structural identification of individual helical amyloid filaments by integration of cryo-electron microscopy-derived maps in comparative morphometric atomic force microscopy image analysis. J. Mol. Biol. 434, 167466 (2022).
10. Y. K. Al-Hilaly, S. J. Pollack, J. E. Rickard, M. Simpson, A.-C. Raulin, T. Baddeley, P. Schellenberger, J. M. D. Storey, C. R. Harrington, C. M. Wischik, L. C. Serpell, Cysteine-independent inhibition of Alzheimer's disease-like paired helical filament assembly by leuco-methylthioninium (LMT). J. Mol. Biol. 430, 4119-4131 (2018).
11. C. M. Wischik, M. Novak, H. C. Thøgersen, P. C. Edwards, M. J. Runswick, R. Jakes, J. E. Walker, C. Milstein, M. Roth, A. Klug, Isolation of a fragment of tau derived from the core of the paired helical filament of Alzheimer disease. Proc. Natl. Acad. Sci. 85, 4506-4510 (1988).
12. C. M. Wischik, P. C. Edwards, R. Y. K. Lai, M. Roth, C. R. Harrington, Selective inhibition of Alzheimer disease-like tau aggregation by phenothiazines. Proc. Natl. Acad. Sci. 93, 11213-11218 (1996).
13. C. R. Harrington, J. M. D. Storey, S. Clunas, K. A. Harrington, D. Horsley, A. Ishaq, S. J. Kemp, C. P. Larch, C. Marshall, S. L. Nicoll, J. E. Rickard, M. Simpson, J. P. Sinclair, L. J. Storey, C. M. Wischik, Cellular models of aggregation-dependent template-directed proteolysis to characterize tau aggregation inhibitors for treatment of Alzheimer disease. J. Biol. Chem. 290, 10862-10875 (2015).
14. R. Lai, C. Harrington, C. Wischik, Absence of a role for phosphorylation in the tau pathology of Alzheimer's Disease. Biomolecules. 6, 19 (2016).
15. C. R. Harrington, E. B. Mukaetova-Ladinska, R. Hills, P. C. Edwards, E. Montejo de Garcini, M. Novak, C. M. Wischik, Measurement of distinct immunochemical presentations of tau protein in Alzheimer disease. Proc. Natl. Acad. Sci. U.S.A 88, 5842-5846 (1991).
16. A. W. P. Fitzpatrick, B. Falcon, S. He, A. G. Murzin, G. Murshudov, H. J. Garringer, R. A. Crowther, B. Ghetti, M. Goedert, S. H. W. Scheres (2017) Cryo-EM structures of tau filaments from Alzheimer's disease. Nature. 547, 185-190.
17. B. Falcon, W. Zhang, A. G. Murzin, G. Murshudov, H. J. Garringer, R. Vidal, R. A. Crowther, B. Ghetti, S. H. W. Scheres, M. Goedert, Structures of filaments from Pick's disease reveal a novel tau protein fold. Nature. 561, 137-140 (2018).
18. Tornquist, M., Cukalevski, R., Weininger, U., Meisl, G., Knowles, T. P. J., Leiding, T., Malmendal, A., Akke, M. and Linse, S. (2020) Ultrastructural evidence for self-replication of Alzheimer-associated AB42 amyloid along the sides of fibrils. Proc. Natl. Acad. Sci., 117, 11265-11273.
19. Frankel, R., Tornquist, M., Meisl, G., Hansson, O., Andreasson, U., Zetterberg, H., Blennow, K., Frohm, B., Cedervall, T., Knowles, T. P. J., Leiding, T. and Linse, S. (2019) Autocatalytic amplification of Alzheimer-associated Aβ42 peptide aggregation in human cerebrospinal fluid. Commun. Biol., 2, 365.
20. Oakley, S. S., Maina, M. B., Marshall, K. E., Al-Hilaly, Y. K., Harrington, C. R., Wischik, C. M. and Serpell, L. C. (2020) Tau filament self-assembly and structure: tau as a therapeutic target. Frontiers Neurol., 11, 1207.
21. Darling, A. L. and Shorter, J. (2020) Atomic structures of amyloid-β oligomers illuminate a neurotoxic mechanism. Trends Neurosci., 43, 740-743.
22. Ciudad, S., Puig, E., Botzanowski, T., Meigooni, M., Arango, A. S., Do, J., Mayzel, M., Bayoumi, M., Chaignepain, S., Maglia, G., Cianferani, S., Orekhov, V., Tajkhorshid, E., Bardiaux, B. and Carulla, N. (2020) Aβ(1-42) tetramer and octamer structures reveal edge conductivity pores as a mechanism for membrane damage. Nature Commun., 11, 3014.
23. Fichou, Y., Al-Hilaly, Y. K., Devred, F., Smet-Nocca, C., Tsvetkov, P. O., Verelst, J., Winderickx, J., Geukens, N., Vanmechelen, E., Perrotin, A., Serpell, L., Hanseeuw, B. J., Medina, M., Buée, L. and Landrieu, I. (2019) The elusive tau molecular structures: can we translate the recent breakthroughs into new targets for intervention? Acta Neuropathol. Commun., 7, 31.
24. Stroud, J. C., Liu, C., Teng, P. K. and Eisenberg, D. (2012) Toxic fibrillar oligomers of amyloid-β have cross-β structure. Proc. Natl. Acad. Sci., 109, 7717-7722.
25. Rickard, J. E., Horsley, D., Wischik, C. M., & Harrington, C. R. (2016). Assays for the Screening and Characterization of Tau Aggregation Inhibitors. In C. Smet-Nocca (Ed.), Tau Protein: Methods and Protocols (Methods in Molecular Biology; Vol. 1523; pp. 129-140). Springer.
26. M. Von Bergen, P. Friedhoff, J. Biernat, J. Heberle, E. M. Mandelkow, E. Mandelkow, Assembly of τ protein into Alzheimer paired helical filaments depends on a local sequence motif (306VQIVYK311) forming β structure. Proc. Natl. Acad. Sci. U.S.A 97, 5129-5134 (2000).
27. Hof, P. R., Bouras, C., Buée, L., Delacourte, A., Perl, D. P. and Morrison, J. H. (1992) Differential distribution of neurofibrillary tangles in the cerebral cortex of dementia pugilistica and Alzheimer's disease cases. Acta Neuropathol. 85, 23-30.
28. McKee, A., Cantu, R., Nowinski, C., Hedley-Whyte, E., Gavett, B., Budson, A., Santini, V., Lee, H.-S., Kubilus, C. & Stern, R. (2009) Chronic traumatic encephalopathy in athletes: progressive tauopathy after repetitive head injury. J. Neuropathol. Exptl. Neurol. 68, 709-735.
30. Wischik, C. M., Theuring, F. and Harrington, C. R. (2001) The molecular basis of tau protein pathology in Alzheimer's disease and related neurodegenerative dementias. In ‘Neurobiology of Alzheimer's Disease’, 2nd Edition, Eds. Dawbarn, D. and Allen, S. J., The Molecular and Cellular Neurobiology Series, Bios Scientific Publishers, Oxford, pp. 103-206.
31. Flament S., Delacourte A. and Mann D. M. A. (1990) Phosphorylation of tau proteins: a major event during the process of neurofibrillary degeneration. A comparative study between AD and Down syndrome. Brain Res., 516, 15-19.
32. Harrington, C. R., Perry, R. H., Perry, E. K., Hurt, J., McKeith, I. G., Roth, M. and Wischik, C. M. (1994) Senile dementia of Lewy body type and Alzheimer type are biochemically distinct in terms of paired helical filaments and hyperphosphorylated tau protein. Dementia 5, 215-228.
33. Hof P. R., Charpiot, A., Delacourte A., Buee, L., Purohit, D., Perl D. P. and Bouras, C. (1992) Distribution of neurofibrillary tangles and senile plaques in the cerebral cortex in postencephalitic parkinsonism. Neurosci. Lett. 139, 10-14.
34. Ikeda K., Akiyama H., Kondo H., Arai T., Arai N. and Yagishita S. (1995) Numerous glial fibrillary tangles in oligodendroglia in cases of subacute sclerosing panencephalitis with neurofibrillary tangles. Neurosci. Lett., 194, 133-135.
35. Love, S., Bridges, L. R. and Case, C. P. (1995) Neurofibrillary tangles in Niemann-Pick disease type C. Brain, 118, 119-129.
36. Ohmi, K., Kudo, L. C., Ryazantsev, S., Zhao, H.-Z., Karsten, S. L. and Neufeld, E. F. (2009) Sanfilippo syndrome type B, a lysosomal storage disease, is also a tauopathy. Proc. Natl. Acad. Sci., 106, 8332-8337.
37. Sergeant, N., Sablonniere, B., Schraen-Maschke, S., Ghestem, A., Maurage, C. A., Wattez, A., Vermersch, P. and Delacourte, A. (2001) Dysregulation of human brain microtubule-associated tau mRNA maturation in myotonic dystrophy type 1. Human Molecular Genetics, 10, 2143-2155 and references cited therein.
38. Maurage, C. A., Udd, B., Ruchoux, M. M., Vermersch, P., Kalimo, H., Krahe, R., Delacourte, A. and Sergeant, N. (2005) Similar brain tau pathology in DM2/PROMM and DM1/Steinert disease. Neurology, 65, 1636-1638.
39. Braak, H., Del Tredici, K, Braak, E. (2003) Spectrum of pathology. In Mild cognitive impairment: Aging to Alzheimer's disease, edited by Petersen, R. C., Oxford University Press; pp. 149-189.
40. Rickard, J. E., Horsley, D., Wischik, C. M., and Harrington, C. R. (2016). Assays for the Screening and Characterization of Tau Aggregation Inhibitors. In C. Smet-Nocca (Ed.), Tau Protein: Methods and Protocols (Methods in Molecular Biology; Vol. 1523; pp. 129-140). Springer.
40. Carlson, H. A. (2022) Protein flexibility and drug design: How to hit a moving target. Curr. Opin. Chem. Biol. 6, 447-452.
41. Cheng, A. C., Coleman, R. G., Smyth, K. T., Cao, Q., Soulard, P., Caffrey, D. R., Salzberg, A. C., Huang, E. S. (2007) Structure-based maximal affinity model predicts small-molecule druggability. Nat. Biotechnol., 25, 71-75.
42. M. Novak, J. Kabat, C. M. Wischik (1993) Molecular characterization of the minimal protease resistant tau unit of the Alzheimer's disease paired helical filament. EMBO J. 12, 365-370.

1. A compound of general formula: 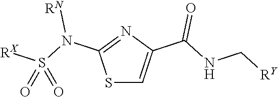
or a pharmaceutically acceptable salt, solvate or hydrate thereof, wherein
R^{N} is independently —H or -Me;
R^{X} is independently C₁₋₄ alkyl, optionally substituted with halo or hydroxy; —NHR^{N1}, wherein R^{N1} is C₁₋₄ alkyl; or C₅₋₁₀ heteroaryl;
R^{Y} is independently: C₅₋₁₀ heteroaryl, optionally substituted with methyl, halo or phenyl; or
neopentyl.

2. A compound according to claim 1, wherein R^{X} is selected from -Me and -Et, optionally substituted with halo or hydroxy.

3. A compound according to claim 2, wherein R^{X} is -Me or -Et, substituted with halo.

4. A compound according to claim 2, wherein R^{X} is -Me substituted with —F or —Cl.

5. A compound according to claim 2, wherein R^{X} is —CH₂F.

6. A compound according to claim 2, wherein R^{X} is -Me or -Et substituted by hydroxy.

7. A compound according to claim 2, wherein R^{X} is —CH₂CH₂OH.

8. A compound according to claim 1, wherein R^{X} is —NHR^{N1}, wherein R^{N1} is C₁₋₄ alkyl.

9. A compound according to claim 8, wherein R^{N1} is -Me or -Et.

10. A compound according to claim 8, wherein R^{X} is —NHMe.

11. A compound according to claim 1, wherein R^{X} is C₅₋₁₀ heteroaryl.

12. A compound according to claim 11, wherein R^{X} is a nitrogen-containing C₅₋₁₀ heteroaryl group.

13. A compound according to claim 12, wherein R^{X} is an indolyl group.

14. A compound according to claim 13, wherein R^{X} is

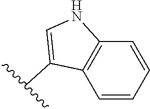 [SMILES: *c1c[nH]c2ccccc12]

15. A compound according to any one of the preceding claims, wherein R^{Y} is neopentyl (—CH₂C(CH₃)₃).

16. A compound according to any one of claims 1 to 15, wherein R^{Y} is C₅₋₁₀ heteroaryl, optionally substituted with methyl, halo or phenyl.

17. A compound according to claim 16, wherein R^{Y} is unsubstituted C₅₋₁₀ heteroaryl.

18. A compound according to claim 17, wherein R^{Y} is a nitrogen-containing C₅₋₁₀ heteroaryl group, optionally substituted with —Cl or phenyl.

19. A compound according to any one of claims 16 to 18, wherein R^{Y} is selected from pyrrolyl, imidazolyl, pyrazolyl, indolyl, and benzimidazolyl.

20. A compound according to claim 19, wherein R^{Y} is selected from:

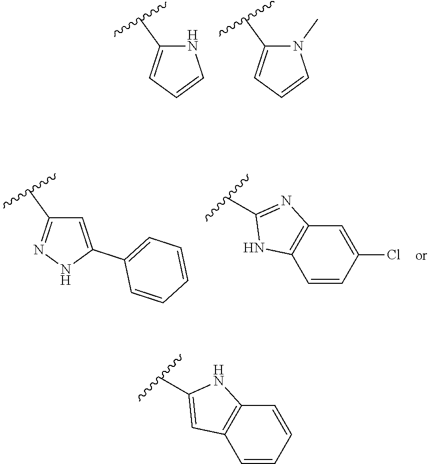

21. A compound according to any one of the preceding claims, wherein R^{N} is —H.

22. A compound according to any one of the preceding claims, wherein R^{N} is -Me.

23. A compound according to claim 1, selected from the following compounds:

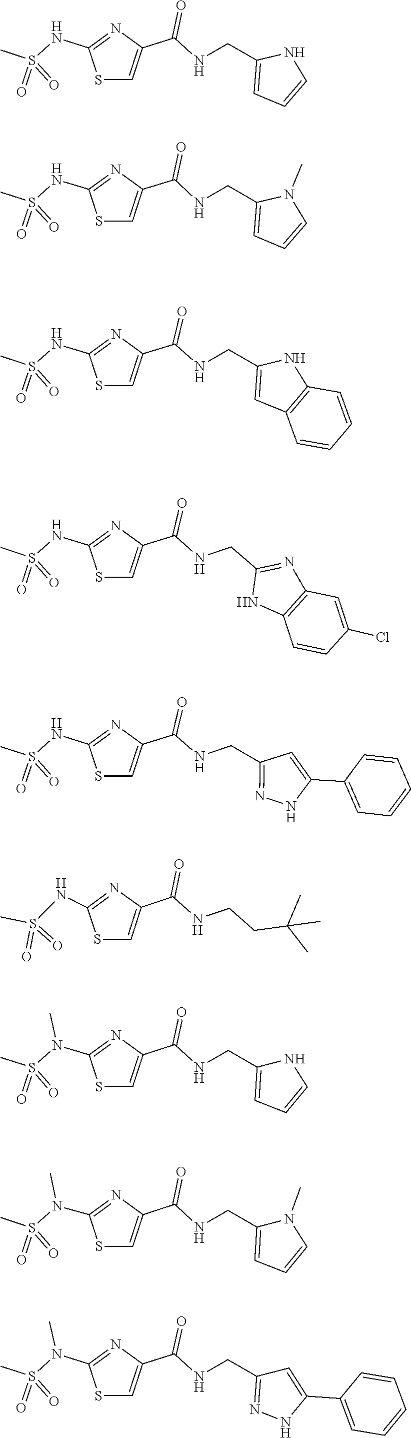

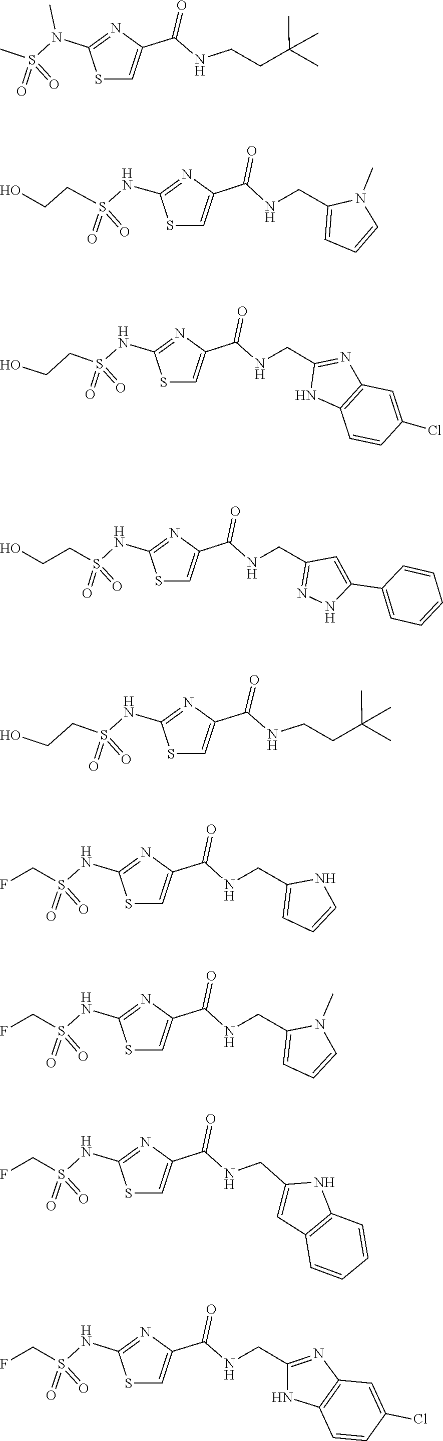

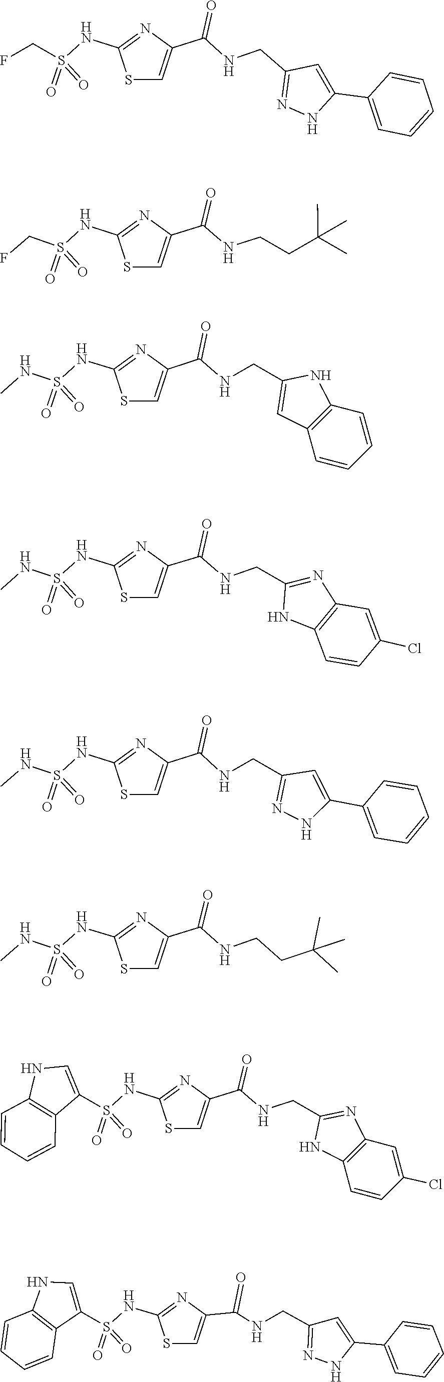

24. A compound according to any one of the preceding claims for use in a method of treatment or prophylaxis of a tauopathy or a disease of tau protein aggregation.

25. A compound for use according to claim 24, wherein the method is for the treatment or prophylaxis of Alzheimer's disease (AD); Pick's disease; Progressive Supranuclear Palsy (PSP); fronto-temporal dementia (FTD); FTD and parkinsonism linked to chromosome 17 (FTDP-17); disinhibition-dementia-parkinsonism-amyotrophy complex (DDPAC); pallido-ponto-nigral degeneration (PPND); Guam-ALS syndrome; pallido-nigro-luysian degeneration (PNLD); cortico-basal degeneration (CBD); dementia with argyrophilic grains (AgD); chronic traumatic encephalopathy (CTE); Down syndrome (DS); dementia with Lewy bodies (DLB); or mild cognitive impairment (MCI).

26. Use of a compound as defined in any one of claims 1 to 23 in the manufacture of a medicament for the treatment or prophylaxis of a tauopathy or a disease of tau protein aggregation.

27. Use according to claim 26, wherein the medicament is for the treatment or prophylaxis of Alzheimer's disease (AD); Pick's disease; Progressive Supranuclear Palsy (PSP); fronto-temporal dementia (FTD); FTD and parkinsonism linked to chromosome 17 (FTDP-17); disinhibition-dementia-parkinsonism-amyotrophy complex (DDPAC); pallido-ponto-nigral degeneration (PPND); Guam-ALS syndrome; pallido-nigro-luysian degeneration (PNLD); cortico-basal degeneration (CBD); dementia with argyrophilic grains (AgD); chronic traumatic encephalopathy (CTE); Down syndrome (DS); dementia with Lewy bodies (DLB); or mild cognitive impairment (MCI).

28. A method of treatment or prophylaxis of a tauopathy or a disease of tau protein aggregation comprising administering a therapeutically effective amount of a compound as defined in any one of claims 1 to 23 to a patient in need thereof.

29. A method according to claim 28, wherein the method is for the treatment or prophylaxis of Alzheimer's disease (AD); Pick's disease; Progressive Supranuclear Palsy (PSP); fronto-temporal dementia (FTD); FTD and parkinsonism linked to chromosome 17 (FTDP-17); disinhibition-dementia-parkinsonism-amyotrophy complex (DDPAC); pallido-ponto-nigral degeneration (PPND); Guam-ALS syndrome; pallido-nigro-luysian degeneration (PNLD); cortico-basal degeneration (CBD); dementia with argyrophilic grains (AgD); chronic traumatic encephalopathy (CTE); Down syndrome (DS); dementia with Lewy bodies (DLB); or mild cognitive impairment (MCI).

30. A pharmaceutical composition for use in the treatment of a tauopathy or a disease of tau protein aggregation, said composition comprising a compound as defined in any one of claims 1 to 23 and a pharmaceutically acceptable excipient, carrier or diluent.
